(19) United States
(12) Patent Application Publication
(10) Pub. No.: US 2013/0103067 A1
(43) Pub. Date: Apr. 25, 2013

(54) DISCECTOMY DEVICES AND RELATED METHODS

(71) Applicants: Myra I. L. Fabro, San Jose, CA (US); Hiep Nguyen, Milpitas, CA (US); Mark Dias, San Jose, CA (US); Al Mirel, Redwood City, CA (US); Robert May, Hayward, CA (US)

(72) Inventors: Myra I. L. Fabro, San Jose, CA (US); Hiep Nguyen, Milpitas, CA (US); Mark Dias, San Jose, CA (US); Al Mirel, Redwood City, CA (US); Robert May, Hayward, CA (US)

(21) Appl. No.: 13/560,850

(22) Filed: Jul. 27, 2012

Related U.S. Application Data
(60) Provisional application No. 61/512,845, filed on Jul. 28, 2011.

(51) Int. Cl. A61B 17/3207

(52) U.S. Cl. CPC A61B 17/3207
USPC 606/170

(57) ABSTRACT

Tissue removal devices are disclosed herein. In some embodiments, a tissue removal device may comprise a handheld housing, a motor, and a tissue removal mechanism coupled to the handheld housing. The tissue removal mechanism may comprise a tubular member, a rotatable elongated member disposed within a lumen of the tubular member, a first impeller distal to the rotatable elongated member, and a second impeller adjacent the first impeller.

This application claims priority to U.S. Provisional Application Ser. No. 61/512,845, entitled “Discectomy Devices and Related Methods,” filed Jul. 28, 2011, which application is incorporated herein by reference in its entirety.

### BACKGROUND

Vertebral disc herniation is a common disorder where a portion of a vertebral disc, a cushion-like structure located between the vertebral bodies of the spine, bulges out or extrudes beyond the usual margins of the disc and the spine. Disc herniation is believed to be the result of excessive loading on the disc in combination with weakening of the annulus due to such factors as aging and genetics. Disc herniation and other degenerative disc diseases are also associated with spinal stenosis, a narrowing of the bony and ligamentous structures of the spine. Although disc herniation can occur anywhere along the perimeter of the disc, it occurs more frequently in the posterior and posterior-lateral regions of the disc, where the spinal cord and spinal nerve roots reside. Compression of these neural structures can lead to pain, parasthesias, weakness, urine and fecal incontinence and other neurological symptoms that can substantially impact basic daily activities and quality of life.

Temporary relief of the pain associated with disc herniation is often sought through conservative therapy, which includes positional therapy (e.g. sitting or bending forward to reduce pressure on the spine), physical therapy, and drug therapy to reduce pain and inflammation. When conservative therapy fails to resolve a patient's symptoms, surgery may be considered to treat the structural source of the symptoms. Surgical treatments for disc herniation traditionally involve open procedures that involve dissection of muscle, connective tissue and bone along a patient's back as well as nerve manipulations to achieve adequate surgical exposure. For example, a discectomy procedure may be used to decompress the herniation by accessing the affected disc and removing a portion of the disc and any loose disc fragments. In some cases, a portion of the lamina or bony arch of the vertebrae may be removed. When discectomy fails to resolve a patient's symptoms, more drastic measures may include disc replacement surgery or vertebral fusion.

### BRIEF SUMMARY

Tissue removal devices and methods are disclosed herein. In some embodiments, a tissue removal device may comprise a handheld housing, a motor, and a tissue removal mechanism coupled to the handheld housing. The tissue removal mechanism may comprise a tubular member, a rotatable elongated member disposed within a lumen of the tubular member, a first impeller distal to the rotatable elongated member, and a second impeller adjacent the first impeller.

In certain embodiments, a tissue removal device may comprise a handheld housing, a motor, and a tissue removal mechanism coupled to the handheld housing. The tissue removal mechanism may comprise a tubular member, a rotatable elongated member disposed within a lumen of the tubular member, an impeller housing coupled to a distal end of the tubular member, and an impeller disposed within the impeller housing and coupled to the rotatable elongated member. The impeller housing may comprise a side wall portion having first and second apertures therethrough, and the first and second apertures may be configured to expose the impeller to tissue during use.

In some embodiments, a tissue removal device may comprise a handheld housing, a motor, and a tissue removal mechanism coupled to the handheld housing. The tissue removal mechanism may comprise a tubular member, a rotatable elongated member disposed within a lumen of the tubular member, a first impeller distal to the rotatable elongated member, and a second impeller adjacent the first impeller.

In certain embodiments, the second impeller may be configured to counter-rotate with respect to the first impeller. In some embodiments, rotation of the rotatable elongated member may effect rotation of the first impeller. Alternatively or additionally, rotation of the first impeller may effect rotation of the second impeller.

The tissue removal device may further comprise a helical member disposed around at least a portion of the rotatable elongated member. Rotation of the rotatable elongated member may effect rotation of the helical member.

In some embodiments, the tissue removal device may further comprise an impeller housing, within which the first and second impellers may be disposed. In certain embodiments, the impeller housing may comprise a side wall portion including a first aperture therethrough. In some embodiments, the side wall portion may further include a second aperture therethrough. The first and second apertures may be configured to expose the first and second impellers to tissue during use. In certain embodiments, at least one of the first and second apertures may define a cutting edge (e.g. having a serrated configuration). The tissue removal device may further comprise a sheath disposed within the impeller housing, and the first and second impellers may be disposed within the sheath.

In some embodiments, the tissue removal device may further comprise a tissue collection chamber coupled to a distal portion of the handheld housing.

In certain embodiments, a tissue removal device may comprise a handheld housing, a motor, and a tissue removal mechanism coupled to the handheld housing. The tissue removal mechanism may comprise a tubular member, a rotatable elongated member disposed within a lumen of the tubular member, an impeller housing coupled to a distal end of the tubular member, and an impeller disposed within the impeller housing and coupled to the rotatable elongated member. The impeller housing may comprise a side wall portion having first and second apertures therethrough. The first and second apertures may, for example, be configured to expose the impeller to tissue during use. In some embodiments, at least one of the first and second apertures may define a cutting edge (e.g. having a serrated configuration). In certain embodiments, the first aperture may be opposite the second aperture along a circumference of the impeller housing.

In some embodiments, a method for treating a spinal disc may comprise advancing one of the above-described tissue removal devices to target disc tissue and removing at least a portion of the target disc tissue with the tissue removal device.

Methods of accessing a target site in a patient are also described here. One embodiment of a method for accessing a target site in a patient may comprise inserting a stylet (e.g. a straight stylet) into a cannula (e.g. a cannula comprising a non-linear configuration), inserting the stylet-cannula assembly into a patient (e.g. where the cannula is at least partially straightened), and removing the stylet from the cannula while substantially maintaining the cannula in the patient. The method may additionally comprise inserting an instrument, such as a tissue removal device, into the cannula.

Methods of accessing a target site in the spine region of a patient are also described here. One embodiment of a method for accessing a target site in the spine region of a patient may comprise inserting a stylet (e.g. a straight stylet) into a cannula (e.g. a curved cannula with a curved distal portion) to form a first cannula-stylet assembly (e.g. with a straight distal portion). The first cannula-stylet assembly may access the spine region, and the stylet may be proximally withdrawn from the first cannula-stylet assembly. A stylet (e.g. a curved stylet with a curved distal portion) may be inserted into the cannula to form a second cannula-stylet assembly (e.g. with a curved distal portion). The second cannula-stylet assembly may be advanced to the target site in the spine region.

Methods for treating a herniated disc are also described here. One embodiment of a method for treating a herniated disc may comprise inserting a stylet (e.g. a straight stylet) into a cannula (e.g. a curved cannula with a curved distal portion) to form a first cannula-stylet assembly (e.g. with a straight distal portion). The first cannula-stylet assembly may penetrate the disc annulus of the herniated disc. The stylet may be proximally withdrawn from the first cannula-stylet assembly, and a stylet (e.g. a curved stylet with a curved distal portion) may be inserted into the cannula to form a second cannula-stylet assembly (e.g. with a curved distal portion). The second cannula-stylet assembly may be advanced to a herniated area. The stylet may be proximally withdrawn from the second cannula-stylet assembly, and a tissue removal device may be inserted into the cannula. A portion of the nucleus pulposus may be removed using the tissue removal device. The tissue removal device may be proximally withdrawn from the cannula, and a stylet (e.g. a straight stylet) may be inserted into the cannula. The stylet and the cannula may be proximally withdrawn.

### BRIEF DESCRIPTION OF THE DRAWINGS

FIG. 1 is a schematic perspective view of a portion of a lumbar spine.

FIG. 2 is a schematic superior view of a portion of a lumbar vertebra and disc.

FIG. 3A is a schematic lateral view of a portion of a lumbar spine (without the spinal nerves), and FIG. 3B depicts the portion of the lumbar spine in FIG. 3A (with the spinal nerves depicted).

FIG. 4A is a side elevational view of a embodiment of a tissue removal device; FIG. 4B is a side elevational view of the tissue removal device of FIG. 4A, with a portion of its housing removed; FIG. 4C is a rear elevational view of the tissue removal device of FIG. 4A; FIG. 4D is a side elevational view of the tissue removal device of FIG. 4A when articulated; FIG. 4E is a side elevational view of the tissue removal device as depicted in FIG. 4D, with a portion of its housing removed; and FIG. 4F is an illustrative cross-sectional view of a portion of an articulation mechanism of the tissue removal device of FIG. 4A.

FIG. 5A is a side elevational view of a shaft of the tissue removal device of FIG. 4A; FIG. 5B is an enlarged view of region 5B of FIG. 5A; FIG. 5C depicts a distal portion of the shaft of FIG. 5A when rotated; and FIG. 5D also depicts a distal portion of the shaft of FIG. 5A when rotated.

FIG. 6A is a side elevational view of an outer tubular member of the shaft of FIG. 5A, and FIG. 6B is an enlarged view of region 6B of FIG. 6A.

FIG. 7A is a side elevational view of an inner tubular member of the shaft of FIG. 5A, and FIG. 7B is an enlarged view of region 7B of FIG. 7A.

FIG. 8A is a side elevational view of a distal region of the shaft of FIG. 5A when rotated, with certain components removed, and FIG. 8B is a side elevational view of a distal tip region of the distal region of the shaft of FIG. 5A when rotated.

FIG. 9 is a side elevational view of a parabolic impeller of the tissue removal device of FIG. 4A.

FIG. 10 is a side elevational view of a tissue removal assembly of the tissue removal device of FIG. 4A.

FIG. 11A is a side elevational view of an embodiment of a shaft of a tissue removal device; FIG. 11B is an enlarged view of region 11B of FIG. 11A; FIG. 11C depicts a distal portion of the shaft of FIG. 11A when rotated; and FIG. 11D also depicts a distal portion of the shaft of FIG. 11A when rotated.

FIG. 12A is a side elevational view of another embodiment of a shaft of a tissue removal device; FIG. 12B is an enlarged view of region 12B of FIG. 12A; and FIG. 12C depicts certain components of a distal portion of the shaft of FIG. 12A, with other components removed.

FIG. 13A is an illustrative view of an additional embodiment of a shaft of a tissue removal device; FIG. 13B is an enlarged view of region 13B of FIG. 13A; FIG. 13C depicts certain components of a distal portion of the shaft of FIG. 13A, with other components removed;

FIGS. 13D and 13E are an illustrative side view and an illustrative top view, respectively, of a distal portion of the shaft of FIG. 13A, with certain components depicted as transparent for visibility of other components; and FIG. 13F is an illustrative cross-sectional view of the distal portion of the shaft as depicted in FIG. 13E, taken along line 13F-13F.

FIG. 14A is an illustrative view of a further embodiment of a shaft of a tissue removal device, and FIG. 14B is an enlarged view of region 14B of FIG. 14A.

### DETAILED DESCRIPTION

Tissue removal devices and methods, such as discectomy devices and methods, are described herein. In certain embodiments, a discectomy device may be introduced into a disc via dilation of an access hole through the annulus, such that it may not be necessary to cut the annulus to access the disc. In some embodiments, a discectomy device may comprise a relatively long auger, and/or an impeller or other member (e.g. that rotates) that breaks down acquired tissue during a procedure. During use, the auger and impeller may effect a plunging motion that allows for relatively rapid tissue aspiration and aggressive tissue cutting, without stretching the annulus. Additionally, it may not be necessary to make several passes into and out of a patient to remove tissue, using devices and methods described herein. By limiting cutting, stretching and/or the number of passes through tissue, scarring of annular tissue, reherniation and/or leakage of healthy nucleus tissue may be avoided, and/or annulus healing time may be reduced.

In some cases, devices described herein may be capable of breaking down soft tissue and/or relatively tough, hardened nucleus tissue, and/or may be used to aspirate different types of tissue varying in consistency, hardness and/or elasticity. In some embodiments, devices described herein may be used to cut hard tissue, such as bone. In some cases, the cant angle of a device's cutting edge or edges (e.g. between the inner base surface of an impeller and the cutting edge of the impeller) may be adjusted to differentially cut relatively hard or calcified materials or tissues without also cutting relatively soft materials or tissues. Examples of these differential cutting heads are described in U.S. Pat. No. 4,445,509, which is hereby incorporated by reference in its entirety.

In some embodiments, to be the least destructive to spine structures while preserving the strength of the bones, a spinal procedure may be minimally invasive while also reducing the amount of excised, native bone or dissection of surrounding native tissues. Minimally invasive tissue removal devices may, for example, be configured for insertion toward or into a vertebral disc without requiring suturing, gluing or other procedures to seal or close the access pathway into the disc. The exemplary embodiments described herein include but are not limited to minimally invasive devices or systems and methods for performing discectomies and other tissue removal procedures, as appropriate. For example, a microdiscectomy may be performed using one or more of the devices and/or methods described herein.

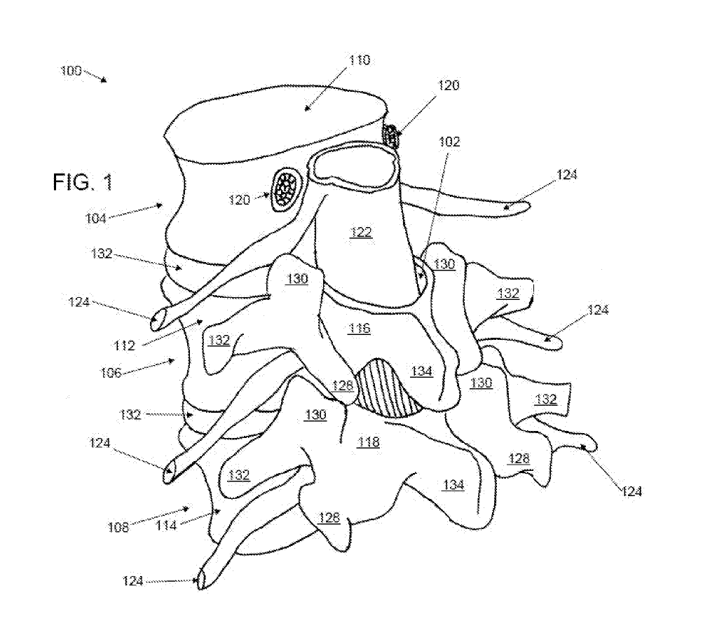

FIG. 1 is a schematic perspective view of a lumbar portion of a spine 100. The vertebral canal 102 is formed by a plurality of vertebrae 104, 106, and 108, which comprise vertebral bodies 110, 112, and 114 anteriorly and vertebral arches 116 and 118 posteriorly. The vertebral arch and adjacent connective tissue of the superior vertebra 104 in FIG. 1 has been omitted to better illustrate the spinal cord 122 within the vertebral canal 102. Spinal nerves 124 branch from the spinal cord 122 bilaterally and exit the vertebral canal 102 through intervertebral foramina 126 that are formed between adjacent vertebra 104, 106 and 108. The intervertebral foramina 126 are typically bordered by the inferior surface of the pedicles 120, a portion of the vertebral bodies 104, 106 and 108, the inferior articular processes 128, and the superior articular processes 130 of the adjacent vertebrae. Also projecting from the vertebral arches 116 and 118 are the transverse processes 132 and the posterior spinous processes 134 of the vertebrae 106 and 108. Located between the vertebral bodies 110, 112 and 114 are vertebral discs 132.

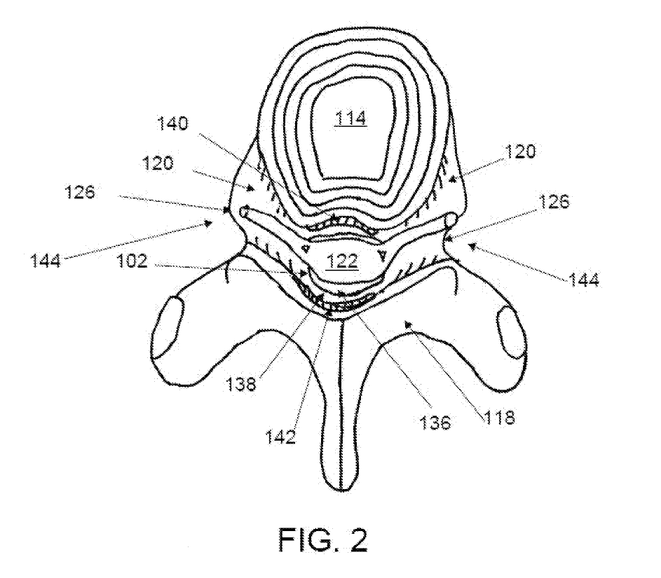

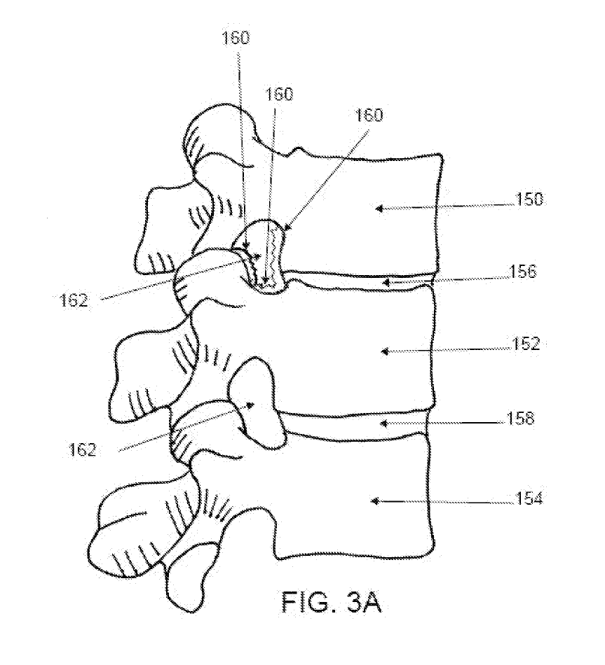

Referring to FIG. 2, the spinal cord 122 is covered by a thecal sac 136. The space between the thecal sac 136 and the borders of the vertebral canal 102 is known as the epidural space 138. The epidural space 138 is bound anteriorly and posteriorly by the longitudinal ligament 140 and the ligamentum flavum 142, respectively, of the vertebral canal 102, and laterally by the pedicles 120 of the vertebral arches 116 and 118 and the intervertebral foramina 126. The epidural space 138 is contiguous with the paravertebral space 144 via the intervertebral foramina 126.

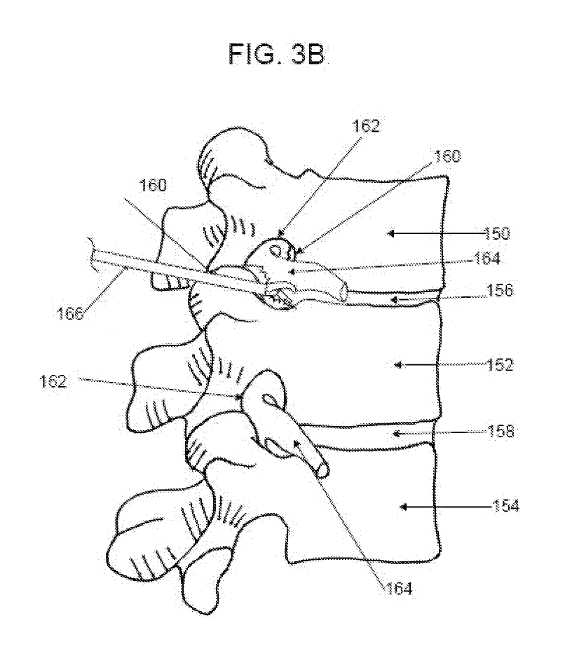

With degenerative changes of the spine, which include but are not limited to disc bulging and hypertrophy of the spinal ligaments and vertebrae, the vertebral canal 102 may narrow and cause impingement of the spinal cord or the cauda equina, a bundle nerves originating at the distal portion of the spinal cord. Disc bulging or bone spurs may also affect the spinal nerves 124 as they exit the intervertebral foramina 126. FIG. 3A, for example, schematically depicts a lateral view of three vertebrae 150, 152 and 154 with intervertebral discs 156 and 158, without the spinal cord or spinal nerves. With degenerative changes, regions of bone hypertrophy 160 may develop about the intervertebral foramina 162. While secondary inflammation of the associated nerve and/or soft tissue may benefit from conservative therapy, the underlying bone hypertrophy remains untreated. The regions of bone hypertrophy 160 may be removed, with or without other tissue, using open surgical spine procedures, limited access spine procedure, percutaneous or minimally invasive spine procedures, or combinations thereof. FIG. 3B depicts the vertebrae 150, 152 and 154 of FIG. 3A with their corresponding spinal nerves 164 during a foraminotomy procedure using a burr or grinder system 166. One example of a limited access spine procedure is disclosed in U.S. Pat. No. 7,108,705, which is hereby incorporated by reference in its entirety. Examples of percutaneous or minimally invasive spine procedures may be found in U.S. Pat. No. 4,573,448, U.S. Pat. No. 6,217,509, and U.S. Pat. No. 7,273,468, which are hereby incorporated by reference in their entirety.

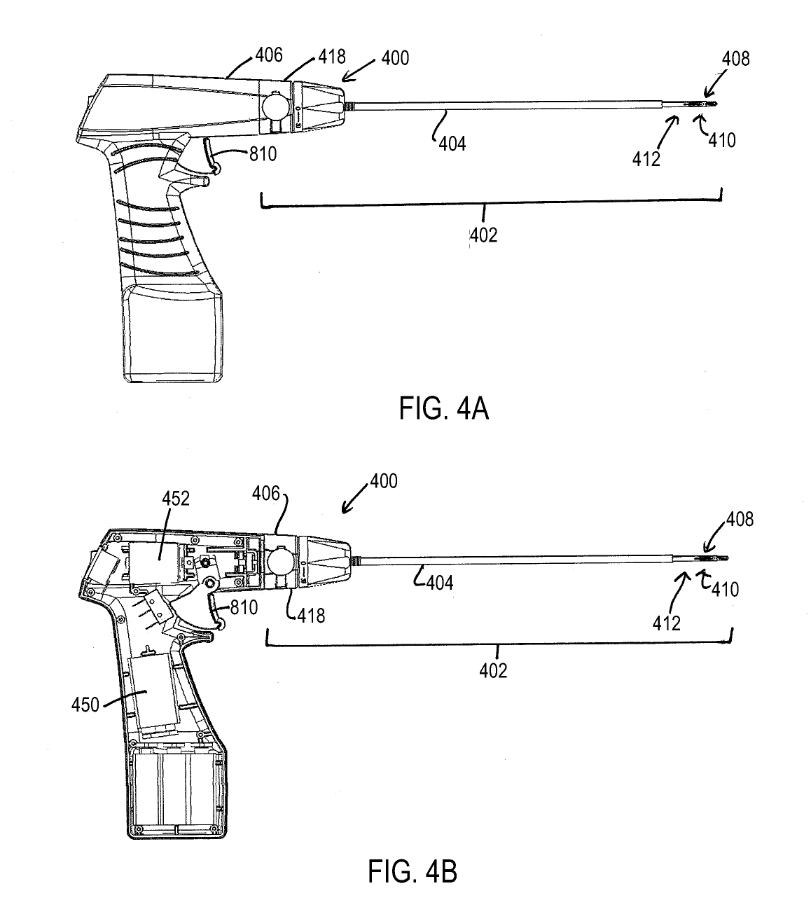

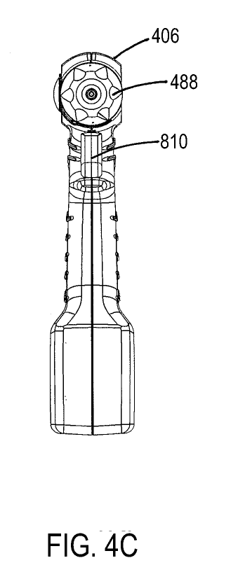

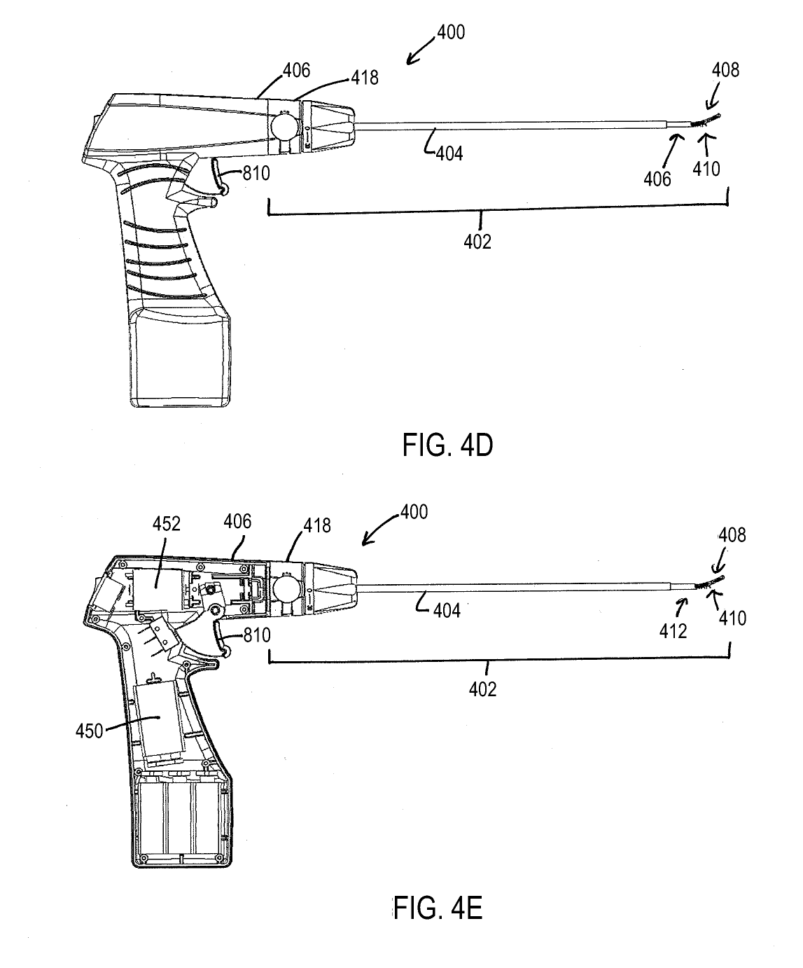

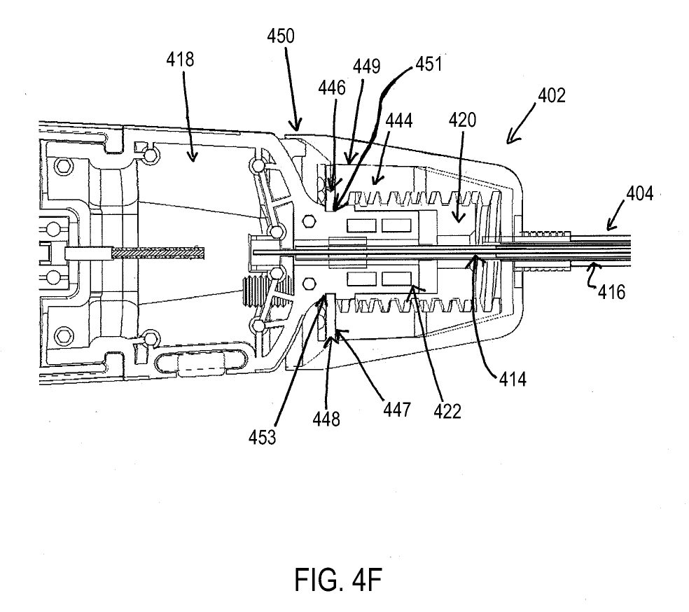

FIGS. 4A-4E depict an exemplary embodiment of a tissue removal device 400 (e.g. a discectomy device). As shown there, tissue removal device 400 comprises an articulation or flexing mechanism 402 comprising a shaft 404. In FIGS. 4A-4C, tissue removal device 400 is in a non-articulating configuration. In other words, the distal portion 412 of shaft 404 is non-articulated or straight. In FIGS. 4D and 4E, tissue removal device 400 is in an articulating configuration, where distal portion 412 is articulated or curved.

The amount of articulation by shaft 404 may be selected, for example, based on the physiology of the patient, the amount of tissue to be removed, and/or the location of the tissue to be removed. In some embodiments, shaft 404 may have a radius of curvature of about 1.00 inch to about 1.75 inches (e.g. about 1.25 inches to about 1.5 inches) when articulated. Alternatively or additionally, shaft 404 may articulate by about 0 degrees to about 30 degrees (e.g. about 1 degree to about 30 degrees, about 5 degrees to about 25 degrees, about 10 degrees to about 15 degrees, about 10 degrees, about 20 degrees, about 30 degrees) relative to its straight or non-articulated configuration. As an example, in some embodiments, shaft 404 may articulate by about 20 degrees to about 25 degrees relative to its straight or non-articulated configuration. The portion of shaft 404 that is capable of articulating may extend along at least about 15% (e.g. at least about 25%) and/or at most about 40% (e.g. at most about 30%) of the length of shaft 404. While the shaft 404 is depicted in FIGS. 4D and 4E as articulating in a particular direction, in other embodiments a shaft may articulate in a different direction.

In some embodiments, the outer diameter of the articulatable portion of the shaft 404 may be smaller than the outer diameter of a proximal, non-articulatable portion of the shaft 404. This may, for example, provide for relatively controlled and/or small dilation of a disc annulus during use. In certain embodiments, the articulatable portion of the shaft 404 may have an outer diameter of about 2 mm to about 3 mm. A proximal, non-articulatable portion of the shaft 404 having a larger outer diameter may remain outside of a disc during use and may, for example, provide support and/or rigidity to the shaft 404. In some embodiments, the portion of the shaft 404 configured for internal disc access during use may have a length of about 2 inches to about 4 inches (e.g. about 3 inches).

Articulation mechanism 402, which is described in further detail below, is configured for an operator to manipulate shaft 404 so that the shaft can be straightened or articulated as desired. The presence of the articulating shaft 404 may allow tissue removal device 400 to remove a greater amount of tissue, and/or to remove more targeted tissue (e.g. targeted disc tissue), than if the system did not include an articulating shaft. For example, in some cases, tissue removal system 400 may be inserted into a patient using the procedure described in U.S. Pat. No. 4,573,448, which is hereby incorporated by reference in its entirety. Articulation mechanism 402 also allows insertion of the device 400 along a straight cannula, but permits deflection from the linear insertion axis after articulation mechanism 402 emerges from the distal end of the cannula. Moreover, articulating shaft 404 may provide an operator with enhanced control over a tissue removal procedure, allowing the operator to adjust the extent of articulation over the course of the procedure. Additionally, in some embodiments, an operator may only need to use a single articulating tissue removal device in a tissue removal procedure, rather than using multiple systems or devices that each have a different amount of curvature. Thus, the number of system or device passes may be reduced which, in turn, may result in a reduction in overall procedure time and cost.

Tissue removal device 400 may be capable of insertion into a disc using relatively small access tubes, as tissue removal device 400 may be inserted in its non-articulated or straight configuration. Examples of an access tube or cannula performed with endoscopic guidance are described in U.S. Patent Application Publication No. US 2010/0121153 A1, while non-endoscopic access to the disc may be performed, for example, using the fluoroscopically guided procedure described in U.S. patent application Ser. No. 12/753,788, both of which are hereby incorporated by reference in their entirety. Once at the target site, articulation mechanism 402 may be actuated, allowing for greater tissue removal than if the device were not capable of articulation. Tissue removal device 400 may be suitable for use in both open surgeries and percutaneous procedures, providing the ability to use a single system for complete disc access through limited spaces. It should be understood from the outset that features and/or characteristics of tissue removal devices and methods described herein may be applied to other tissue removal devices and methods (including others described herein), as appropriate. Moreover, a tissue removal device may include any suitable combination of the features described herein.

Referring again to FIGS. 4A-4E, device 400 further comprises a handle housing 406 that is coupled to shaft 404. Additionally, tissue removal device 400 comprises a tissue removal assembly 408 in its distal portion 410. Tissue removal assembly 408 may be activated prior to, during, and/or after articulation of shaft 404.

The housing 406 contains one or more components configured to control the tissue removal assembly 408 and other optional features of the tissue removal device 400. In some cases, the housing 406 may comprise a control interface that may be used to control the power state of the tissue removal device 400, including but not limited to on and off states. The control interface may, for example, comprise a trigger that may be squeezed to operate the device and/or may comprise a push button, a slide, a dial, a knob, a lever and/or a pivot member, for example. In some embodiments, the control interface may also change the motor speed and/or movement direction of the tissue removal assembly 408. A bi-directional tissue removal device may be provided, for example, as a potential safety feature should the tissue removal assembly 408 get lodged in a body tissue or structure. The control interface may be analog or digital, and may comprise one or more detent positions to facilitate selection of one or more pre-selected settings. In other embodiments, a separate motor control interface may be provided for one or more features of the motor. In still other embodiments, control interfaces for other features of the tissue removal device may be provided.

Referring specifically to FIG. 4C, the handle housing 406 may include a trigger 810, the other side of which is shown in FIGS. 4A, 4B, 4D and 4E. Additionally, the handle housing 406 may include an articulating knob 488 which may serve as an interface that allows the operator to control the extent of articulation of the shaft 404. During use, an operator may hold the handle housing 406 with one hand and may use his or her other hand to rotate the articulating knob 488 (e.g. clockwise). This rotation may result in flexing or articulation of the shaft 404. In some embodiments, the articulating knob 488 may be marked (e.g. pad printed) with articulation angle options, so that the operator may relatively easily select the desired extent of shaft articulation. In certain embodiments, the operator may make this selection by aligning an arrow printed on the top of a collection chamber 418 (described in further detail below) with printing on the collection chamber indicating the resulting articulation angle. The articulating knob 488 may also include an internal tab or other feature that provides a stop at a selected maximum shaft articulation. For example, if the desired maximum articulation angle is 30 degrees, then the internal tab may stop the articulating knob 488 from rotating further once the articulation angle has reached 30 degrees. In some embodiments, the operator may decrease the articulation angle or straighten the shaft 404 by rotating the articulating knob 488 in the opposite direction (e.g. counterclockwise).

The handle housing 406 may further comprise one or more ridges, recesses and/or sections of textured or frictional surfaces, including but not limited to styrenic block copolymers or other polymer surfaces. Although not depicted in FIG. 4C, tissue removal device 400 may optionally comprise a travel limiter mechanism. Travel limiter mechanisms are disclosed, for example, in U.S. Provisional Patent Application Ser. No. 61/413,925, which is hereby incorporated by reference in its entirety.

Tissue removal assemblies such as tissue removal assembly 408 may be configured to grasp, cut, chop, grind, burr, pulverize, debride, debulk, emulsify, disrupt or otherwise remove tissue, as appropriate. Emulsification includes, for example, forming a suspension of tissue particles in a medium, which may be the existing liquid at the target site, liquid added through the tissue removal device, and/or liquid generated by the debulking of the tissue. Optional components of tissue removal device 400 and other tissue removal devices described herein may include, but are not limited to, a motor configured to rotate or move one or more components of the tissue removal assembly, a power source or power interface, a motor controller, a tissue transport assembly (e.g. comprising an auger), an energy delivery or cryotherapy assembly, a therapeutic agent delivery assembly, a light source, and one or more fluid seals. The optional tissue transport assembly may comprise a suction assembly and/or a mechanical aspiration assembly. For example, the tissue removal device 400 may further comprise an optional port that may be attached to an aspiration or suction source to facilitate transport of tissue or fluid out of the target site or patient. The suction source may be a powered vacuum pump, a wall suction outlet, or a syringe, for example.

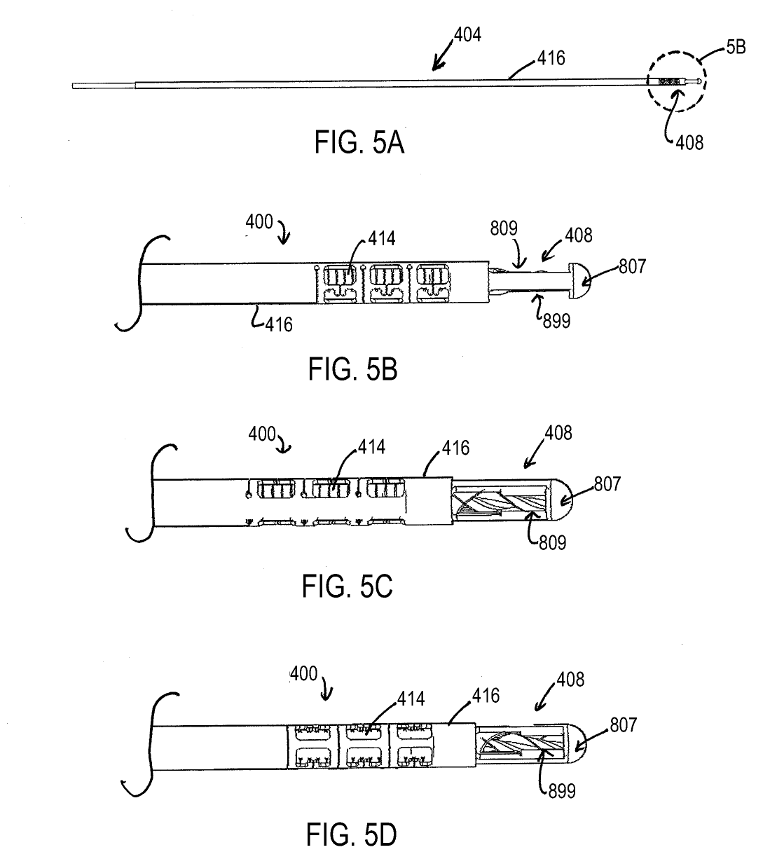

Referring now to FIGS. 5A-5D, 6A, 6B, 7A and 7B, shaft 404 comprises an inner tubular member 414 disposed within an outer tubular member 416. The inner and outer tubular members are coupled to each other (e.g. soldered, snap-locked, etc.) at their distal ends. Articulation may be achieved by effectively pulling on outer tubular member 416 while maintaining the relative position of inner tubular member 414, and thereby causing the inner and outer tubular members to bend. In other embodiments of tissue removal devices, articulation may be achieved by pulling on an outer tubular member while pushing on an inner tubular member (or vice versa), or by pushing on an inner tubular member or an outer tubular member.

Referring as well now to FIG. 4F, in addition to comprising shaft 404, articulation mechanism 402 comprises a collection chamber 418 (also shown in FIGS. 4A, 4B, 4D and 4E) configured to collect tissue that is transported proximally along the shaft. Collection chamber 418 may comprise any appropriate material or materials, such as one or more polymers (e.g. polycarbonate) and may be formed using, for example, an injection molding process.

Articulation mechanism 402 further comprises an outer tubular member capture drum 420 coupled to outer tubular member 416 (e.g. by one or more adhesives) and configured to slide along cylindrical member 422 of collection chamber 418. Drum 420 may comprise any appropriate material or materials, including but not limited to metals, metal alloys (e.g. stainless steel) and polymers (e.g. polycarbonate), and may be formed using any appropriate process, such as screw-machining or molding (e.g. injection molding).

Cylindrical member 422 has one or more (e.g. two) protrusions configured to index or engage with one or more (e.g. two) corresponding grooves and/or slots in drum 420. The protrusions may, for example, be generally in the shape of right-angled parallelepipeds or any other suitable shape, and may extend along the length of cylindrical member 422 or along a lesser distance. As a result of the indexing between cylindrical member 422 and drum 420, drum 420 is capable of sliding back and forth on cylindrical member 422, but is not capable of rotating around the cylindrical member. While cylindrical member 422 comprises one or more protrusions and drum 420 comprises one or more corresponding grooves and/or slots, in other embodiments of systems, a cylindrical member may alternatively or additionally comprise one or more grooves and/or slots, and/or a drum may alternatively or additionally comprise one or more protrusions. Moreover, a cylindrical member and drum may also be engaged or indexed to each other in one or more other ways.

Articulation mechanism 402 further comprises an articulation drive 444 coupled to both drum 420 and cylindrical member 422. Articulation drive 444 may comprise any suitable material or materials, including but not limited to metals, metal alloys (e.g. stainless steel) and polymers (e.g. polycarbonate), and may be formed using any appropriate process, such as screw-machining or molding (e.g. injection molding). Articulation drive 444 has internal female threading and drum 420 has corresponding external male threading, such that the drum and the articulation drive can be coupled to each other. Additionally, articulation drive 444 is coupled to cylindrical member 422 via locking tabs 446 and 448. Locking tabs 446 and 448 are configured to fit through slots 447 located in a proximal portion 449 of articulation drive 444, as well as within grooves 451 located in a proximal portion 453 of cylindrical member 422. Locking tabs 446 and 448 may be used to prevent axial movement of articulation drive 444 (i.e. to prevent articulation drive 444 from translating), while allowing articulation drive 444 to rotate and thereby drive drum 420 forward (distally) and backward (proximally).

As shown, articulation mechanism 402 further comprises an optional articulation knob 450 (e.g. that is ergonomically designed). Articulation knob 450 is located distal to collection chamber 418, and covers cylindrical member 422, drum 420 and articulation drive 444 during use. Articulation knob 450 may comprise any appropriate material or materials, such as one or more polymers (e.g. polycarbonate) and may be formed using, for example, an injection molding process.

In operating articulation mechanism 402, an operator may articulate shaft 404 by rotating articulation knob 450. This causes articulation drive 444 to rotate and drum 420 to translate distally or proximally. The proximal translation of drum 420 pulls on outer tubular member 416 and thereby effects articulation of shaft 404. The operator may adjust the amount of articulation of shaft 404 throughout a procedure by adjusting articulation knob 450. In some embodiments, articulation knob 450 may comprise one or more features that prevent over-rotation of the articulation knob. As an example, articulation knob 450 may include one or more tabs and/or other stop elements configured to contact a corresponding element on collection chamber 418 (e.g. after the knob has been rotated clockwise by a certain amount). An articulation knob may be configured to rotate clockwise, counterclockwise, or in both directions.

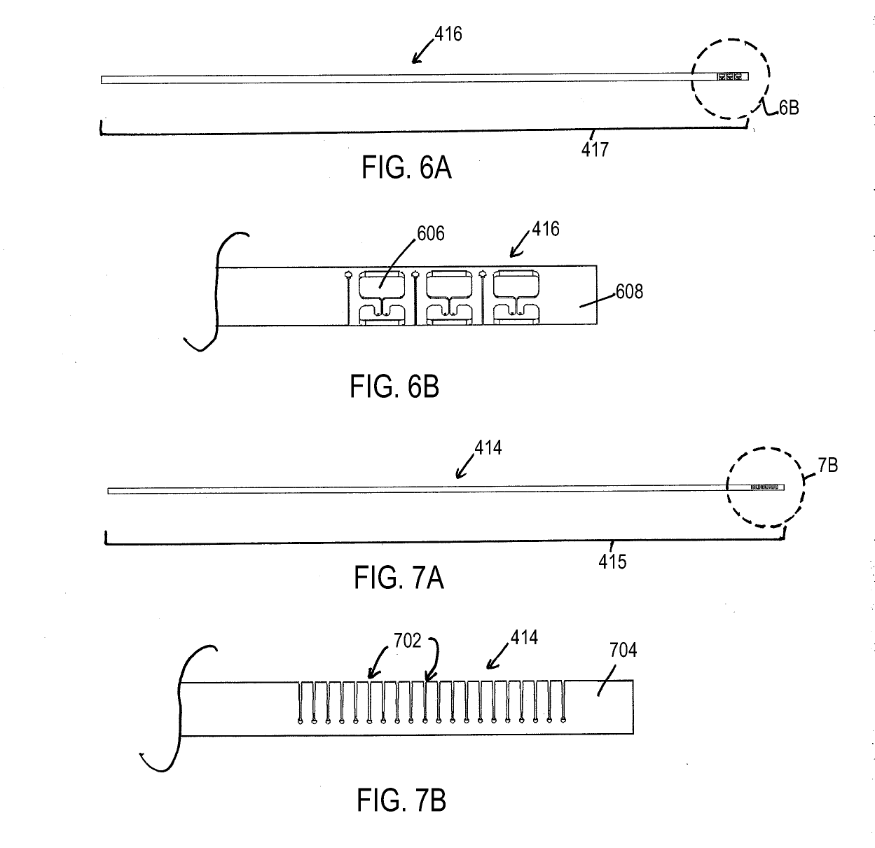

FIGS. 5A-5D depict inner and outer tubular members 414 and 416, as well as tissue removal assembly 408. Additionally, FIGS. 6A and 6B provide depictions of the outer tubular member 416, and FIGS. 7A and 7B provide depictions of the inner tubular member 414. As shown in FIGS. 7A and 7B, inner tubular member 414 has a length 415. Additionally, as shown in FIGS. 6A and 6B, outer tubular member 416 has a length 417. In some embodiments, length 415 and/or length 417 may be from about 17 centimeters to about 36 centimeters (e.g. about 20 centimeters to about 27 centimeters, about 22 centimeters to about 24 centimeters). A relatively long length 415 or 417 may be selected, for example, for use with an endoscopic system. As depicted, length 415 may be greater than length 417 (e.g. to allow inner tubular member 414 to extend into collection chamber 418), although in other embodiments, different dimensions may be employed.

Inner and outer tubular members 414 and 416 may comprise the same material or materials, or may comprise different materials. Examples of materials which may be suitable for inner tubular member 414 and/or outer tubular member 416 include metals and metal alloys, such as stainless steel. In some embodiments, the material or materials used for the inner and/or outer tubular members may be selected to achieve a desirable balance between stiffness and flexibility.

As shown in FIGS. 7A and 7B, inner tubular member 414 comprises slits 702 in its wall portion 4604. Additionally, as shown in FIGS. 6A and 6B, outer tubular member 416 comprises apertures 606 in its wall portion 608. During use, when articulation knob 450 is rotated, inner and outer tubular members 414 and 416 may translate relative to each other, thereby resulting in an overall articulation of shaft 404. Slits 702 may, for example, allow inner tubular member 414 to maintain good rigidity and shape, even when articulated. As a result, shaft 404 may be unlikely to deflect upon contacting bone, for example.

Slits 702 and/or apertures 606 may be formed using any appropriate method, such as laser-cutting. The slits and apertures may be of any suitable size and/or shape, and in some embodiments, combinations of different sizes and/or shapes may be used. In certain embodiments, one or more of slits 702 may have an unwrapped (or straight) length along the circumference of the inner tubular member 414 of about 0.150 inch to about 0.180 inch. The unwrapped length of a slit 702 may be, for example, about 60% to about 70% of the circumference of the inner tubular member 414. Such a ratio may, for example, provide for sufficient flexibility in that portion of the inner tubular member 414, while also maintaining its strength. The remaining uncut circumference may act as a strut, for example. In some embodiments, one or more of slits 702 may have a kerf or cut width of about 0.002 inch to about 0.020 inch (e.g. about 0.005 inch to about 0.015 inch). Alternatively or additionally, one or more of apertures 606 may have a length of about 0.300 inch to about 0.500 inch (e.g. about 0.320 inch to about 0.360 inch) and/or a wrapped dimension (i.e. a measurement taken along the circumference of outer tubular member 416) of about 0.050 inch to about 0.090 inch.

While certain embodiments of articulating tissue removal devices or systems have been described, other embodiments may alternatively or additionally be used in a tissue removal procedure. Articulating tissue removal devices or systems may have at least one of the features described herein, and/or one or more other features. As an example, in some embodiments an articulating tissue removal system may comprise a shaft (e.g. formed of a metal alloy such as Nitinol) having a curved distal portion. The system may further comprise a straight tubular member such as a sheath or overtube (e.g. formed of a metal alloy such as stainless steel) that may be distally advanced over the shaft to incrementally straighten the distal portion of the shaft. In certain embodiments, the straight tubular member may also be configured for proximal translation, so that the extent of shaft articulation may be adjusted as desired during a procedure. The straight tubular member may, for example, be coupled to an actuation mechanism, such as a rotatable knob, that may be used to advance the straight tubular member. Other embodiments of articulating tissue removal systems and devices may also be employed in a tissue removal procedure.

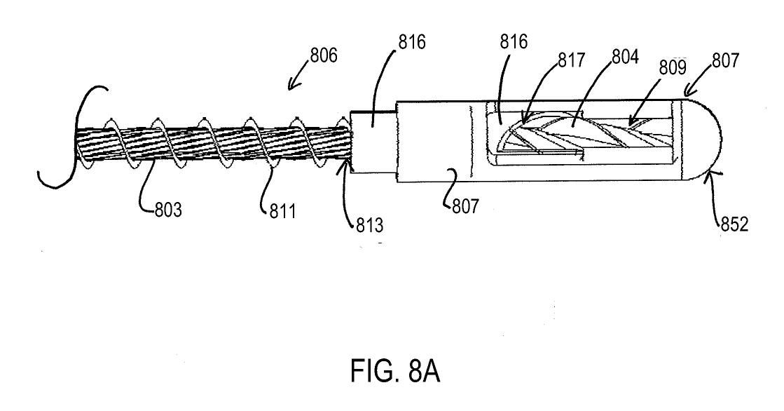

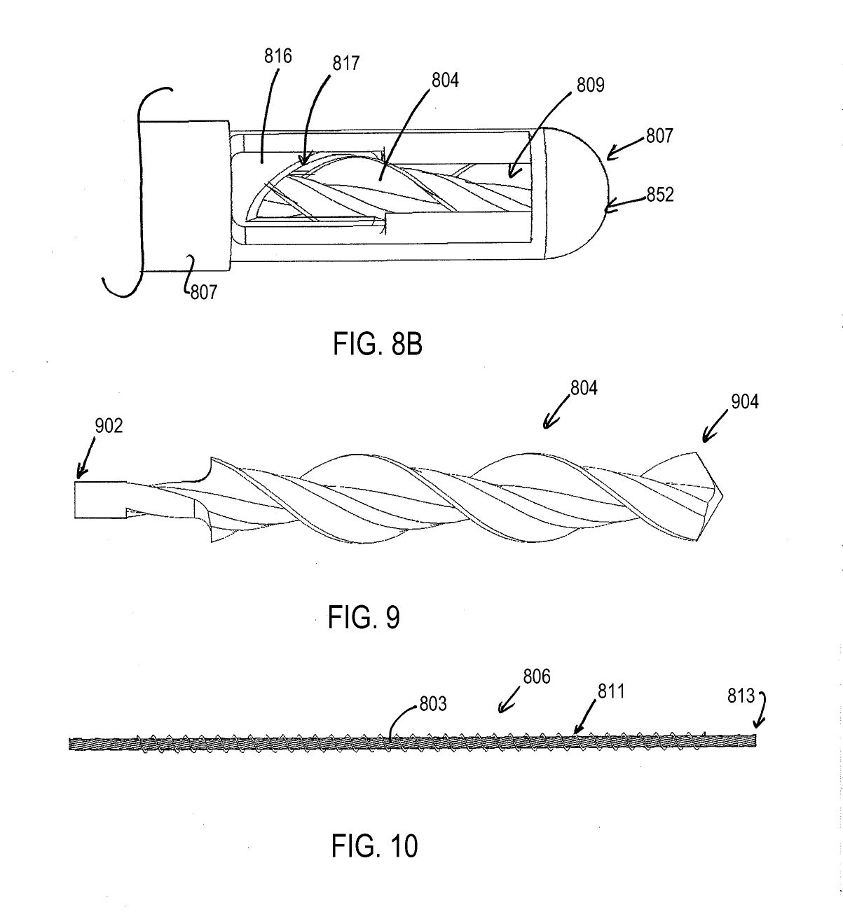

As discussed above, the device 400 comprises a tissue removal assembly 408. Referring now to FIGS. 8A and 10, the tissue removal assembly 408 may comprise a tissue transport assembly 806 comprising a drive shaft 803 and a helical member or auger 811 coupled to the drive shaft 803. Additionally, and referring now to FIGS. 8A, 8B and 9, the tissue removal assembly 408 may comprise an impeller 804 that may be coupled (e.g. welded) to a distal end 813 of drive shaft 803. Tissue removal assembly 408 may further comprise a hooded tip or impeller housing 807 having at least one aperture—as shown in FIGS. 5B-5D, two apertures 809 and 899. In other embodiments of devices, more than two (e.g. three, four, five, ten) apertures may be used, as appropriate. In devices comprising multiple apertures, at least some of the apertures may have the same size and/or shape, and/or at least some of the apertures may have different sizes and/or shapes. Impeller 504 is disposed within hooded tip 807, but is partially exposed as a result of apertures 809 and 899. The presence of apertures 809 and 899 within hooded tip 807 may, for example, increase or otherwise enhance tissue removal efficiency by allowing the impeller 504 to contact tissue from two different (here, opposing) sides of the tissue removal device 400. In some cases, additional apertures may be employed to further enhance impeller exposure. In cases in which multiple apertures are used in a hooded tip, the apertures may or may not form a pattern and/or may have the same or different sizes. Apertures may or may not be connected, and may have any suitable orientation or location relative to each other (e.g. 180 degrees apart from each other, 90 degrees apart from each other, etc.).

In some cases, and referring to FIGS. 8A and 8B, the tissue removal assembly 408 may comprise a sheath 816 at least partially disposed within the hooded tip 807 and comprising a shearing edge 817. The shearing edge 817 may, for example, provide for additional tissue scraping and/or removing capabilities (e.g. without increasing the likelihood of unintentionally perforating a disc annulus or gouging an end-plate). The tissue removal edges of a sheath or other components of the tissue removal device 400 may act in conjunction with impeller 804, or may be used by the operator to manually remove tissue (e.g. manipulating the sheath 816 in a back and forth motion to remove tissue). While sheath 816 is depicted as having one shearing edge 817, other embodiments of sheaths may have multiple shearing edges or may have shearing edges that are oriented differently (e.g. at a different angle relative to a longitudinal axis of the impeller). For example, a sheath may have an opening including a V-shape that provides two shearing edges.

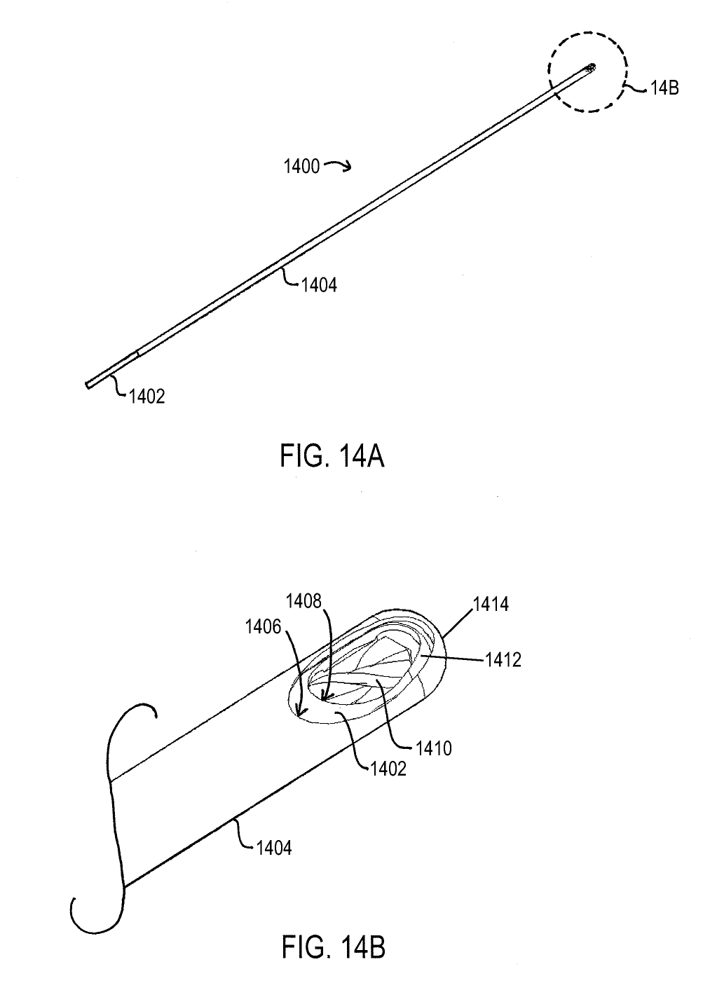

In some cases, a tissue removal device may comprise a distal sheath (e.g. sheath 816) or other component (e.g. an inner tubular member), that is configured to rotate within a hooded tip or housing or other component (e.g. an outer tubular member), while a shaft and/or auger of the tissue removal device remain static. As an example, FIGS. 14A and 14B show a tissue removal device shaft 1400 comprising an inner tubular member 1402 partially disposed within an outer tubular member 1404. The inner and outer tubular members 1402 and 1404 comprise rounded distal end portions 1412 and 1414, respectively. Additionally, the inner tubular member 1402 has a distal opening 1408, and the outer tubular member 1404 has a distal opening 1406. The distal openings 1406 and 1408 expose other components, such as an impeller 1410 and/or an auger (not shown), to tissue (e.g. when the distal openings are aligned). In some embodiments, at least one of the distal openings 1406 and 1408 may include one or more features, such as serrations, which may assist with tissue cutting or removal.

While the inner and outer tubular members of FIGS. 14A and 14B are depicted as each having one distal opening, in other embodiments, a shaft may comprise an inner tubular member with more than one distal opening and/or an outer tubular member with more than one distal opening.

In certain embodiments, the auger (not shown) may not rotate during use. For example, the auger may comprise an extension that is coupled (e.g. bonded) to a non-rotating member in a handle of the tissue removal device. The outer tubular member 1404 may also not rotate during use. For example, the outer tubular member 1404 may be coupled (e.g. bonded) to a non-rotatable collection chamber of a handle of the tissue removal device. Additionally, the inner tubular member 1402 may rotate during use. For example, the inner tubular member 1402 may be coupled to a rotating driveshaft of the tissue removal device. When the tissue removal device is operated, the impeller 1410 may “grab” tissue and the rotating inner tubular member 1402 may shave the tissue against the inside of the static outer tubular member 1404. While a certain tissue shaving configuration has been described, other configurations may be employed as appropriate. As an example, multiple rotating components may be employed in some embodiments.

The result may be that the edges of the rotating sheath tip “shave” tissue as they come into contact with an edge of the hooded tip or housing. In certain embodiments, such a tissue removal device may not comprise any impellers or other cutters, aside from the rotating sheath. In such embodiments, the absence of impellers or other cutters may provide for more space within a distal hooded tip or housing, within which to collect tissue. Alternatively, a moving sheath may in some cases be used in conjunction with one or more impellers and/or other cutters. Additionally, while rotating sheaths have been described, in certain embodiments a sheath may move in a different way. For example, a sheath may translate along a longitudinal axis of a tissue removal device. In some embodiments, a sheath may both rotate and translate. This may, for example, provide for highly targeted tissue removal.

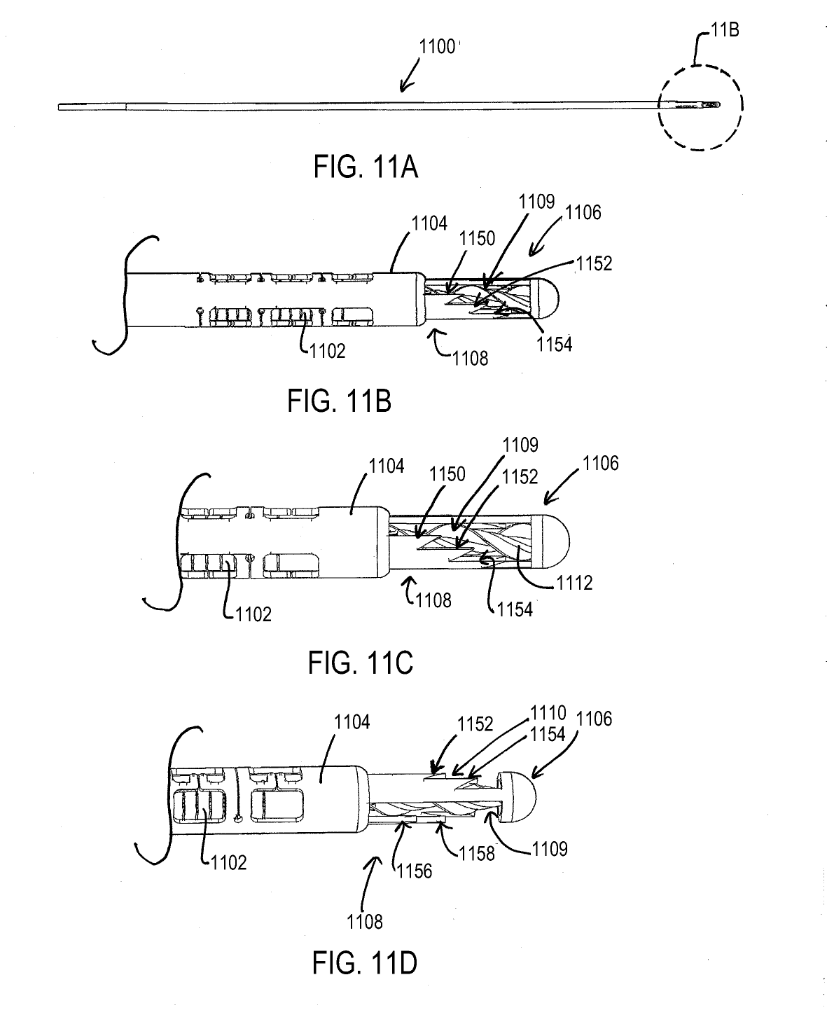

While certain embodiments of shearing or cutting edges have been described, other embodiments of shearing or cutting edges may be employed in a tissue removal device. Shearing or cutting edges may have any appropriate size, shape or configuration. For example, FIGS. 11A-11D show a tissue removal device having multiple shearing edges. As shown there, a shaft 1100 of a tissue removal device, such as a discectomy device, includes an inner tubular member 1102 and an outer tubular member 1104. The tissue removal device further comprises a tissue removal assembly 1106 in a distal portion 1108 of the shaft 1100. Tissue removal assembly 1106 comprises a hooded tip 1107 having at least one aperture—as shown in FIGS. 11B-11D, two apertures 1109 and 1110. An impeller 1112 is disposed within hooded tip 1107, but is partially exposed as a result of the presence of apertures 1109 and 1110. As previously described, the presence of multiple apertures in the hooded tip may provide for enhanced tissue removal (e.g. because of greater exposure of the impeller 1112 to the tissue). In some cases, at most about 50% (e.g. at most about 40%) and/or at least about 20% (e.g. at least about 30%) of the surface area of the impeller 1112 may be covered by the hooded tip 1107. Alternatively or additionally, at most about 80% (e.g. at most about 60%) and/or at least about 30% (e.g. at least about 50%) of the circumference of the impeller 1112 may be covered by the hooded tip 1107. The degree of exposure of the impeller 1112 may be selected, for example, based on the desired extent and/or rate of tissue removal.

As shown in FIGS. 11B-11D, the hooded tip 1107 itself includes multiple shearing edges 1150, 1152, 1154, 1156 and 1158. These edges form a shape that is similar to a serrated pattern. However, other suitable shapes and patterns may be used. In FIGS. 11B-11D, the tissue removal assembly 1106 does not include a separate sheath having a shearing edge. However, while devices with sheaths comprising a shearing edge and devices with hooded tips or housings comprising multiple shearing edges have been depicted, other embodiments of devices may comprise any suitable arrangement and configuration of shearing edges, such as a sheath comprising multiple shearing edges, a hooded tip comprising just one shearing edge, or both a sheath comprising one or more shearing edges and a hooded tip comprising one or more shearing edges. Moreover, in some cases a hooded tip with one or more shearing edges may be used with a sheath that does not have any shearing edges.

In certain cases, a hooded tip or housing (e.g. the edge of an opening or aperture of a hooded tip) may include one or more other features, such as grooves, channels, sharpened or serrated configurations, or the like, that may be used to further enhance tissue cutting and maceration (e.g. by acting as a static cutting edge). In some cases, the interior surface of a hooded tip or housing may alternatively or additionally comprise one or more protrusions, recesses and/or other cutting structures to facilitate further tissue disruptions.

Referring again to FIGS. 8A and 8B, when tissue removal device 400 is operated, auger 811 may advance into and retract from inner tubular member 414, with impeller 804 acting as a forward “drilling” cutter and also in some cases as a side cutter (e.g. as a result of the impeller's interactions with the hooded tip 807). Auger 811 may be actuated, for example, by pulling on trigger 810 of the device 400 (FIGS. 4A-4E). Other suitable actuation mechanisms, such as a switch or a push-button mechanism, may also be used.

Auger 811 may be used to facilitate transport or removal of tissue within or along the shaft 404. In the particular embodiment depicted, auger 811 is mounted on rotatable drive shaft 803, and is also capable of moving axially. Actuation of the rotatable shaft 803 may mechanically facilitate proximal movement of tissue or other materials within a channel or lumen of the shaft 404 by rotating auger 811. The actuated rotatable shaft 803 will also rotate impeller 804. In some embodiments, use of tissue transport assembly 806 may be performed at lower rotational speeds when tissue debulking is not concomitantly performed. When rotated in the opposite direction, the auger 811 may be used expel or distally transport tissue, fluid or other materials or agents from the shaft 404 or supplied to an infusion port of the housing 406.

In some embodiments, auger 811 may have a longitudinal dimension of about 6 inches to about 15 inches (e.g. about 6 inches to about 12 inches, such as about 8 inches to about 10 inches). In other embodiments, the longitudinal dimension of the auger 811 may be characterized as a percentage of the longitudinal dimension of the shaft 404 (e.g. the inner tubular member 414 and/or the outer tubular member 416), and may range from about 5% to about 100% of the longitudinal dimension, sometimes about 10% to about 50%, and other times about 15% to about 25%, and still other times about 5% to about 15%. Although the auger 811 depicted in FIG. 10 rotates at the same rate as the rotatable drive shaft 803 and the impeller 804, in other embodiments, an auger may be configured to rotate separately from other device components. For example, an auger may comprise a helical coil that is located along at least a proximal portion of the lumen of the outer tube but is not mounted on a rotatable shaft. In this particular example, the auger may rotate independently from a shaft (e.g. a rotatable shaft).

Although auger 811 is depicted as a continuous structure, in some embodiments, auger 811 may be interrupted at one or more locations. Also, the degree or angle of tightness of the auger 811 may vary, from about 0.5 turns/mm to about 2 turns/mm, sometimes about 0.75 turns/mm to about 1.5 turns/mm, and other times about 1 turn/mm to about 1.3 turns/mm. The cross-sectional shape of the auger 811 may be generally rounded, but in other embodiments, may have one or more edges. The general cross-sectional shape of the auger 811 may be circular, elliptical, triangular, trapezoidal, squared, rectangular or any other shape. The turn tightness and cross-sectional shape or area of the auger 811 may be uniform or may vary along its length. In some embodiments, multiple augers may be provided in parallel or serially within the outer tubular member.

A tissue removal device with a flexible region may facilitate access to certain regions of the body, such as the central spinal canal through an intervertebral foramen. During use, the tissue removal assembly 408 may, for example, be introduced into a disc via dilation through the annulus, such that no annular tissue is cut. The tissue removal assembly 408 and any other device components that pass through the annular tissue may have a maximum outer diameter of, for example, about 2 mm to about 4 mm (e.g. about 3 mm to about 4 mm). This may allow the access hole to be dilated to a size where healing and sealing of the annulus can occur more easily than may be the case with a cut annulus.

Tissue removal device 400 may allow for a relatively significant extension of auger 811, with impeller 804 working at grabbing and aspirating tissue. The impeller 804 may work to break down acquired tissue in conjunction with one or more shearing or cutting edges, such as the shearing edge 817. The device 400 may not stretch the disc annulus, as passes of the tissue removal mechanism may not be required. For example, the auger 811 may effectively plunge back and forth so that the impeller 804 may drill through the target tissue. This auger/impeller “plunging” may allow for relatively quick tissue aspiration and/or additional significant cutting into the tissue.

The hooded tip or housing 807 of tissue removal assembly 408 comprises a rounded distal head 852, as shown in FIGS. 8A and 8B. This rounded distal head may serve as a guard that, for example, prevents inadvertent cutting into a vertebral end-plate or an annulus during use. Additionally, the internal edge of the rounded distal head may comprise a chamfer. The chamfer may, for example, provide a controlled scraping edge that can act as a curette and help pull disc nucleus toward impeller 804 without cutting into a vertebral end-plate or an annulus.

While tissue removal assembly 408 comprises a rounded distal head 852, other head configurations are also contemplated, including but not limited to a conical configuration, an ovoid configuration, a dome configuration, a concave configuration, a cube configuration, etc. The head 852 may be configured to penetrate or dissect body tissue, such as the annular wall of a vertebral disc, and may be used while the rotatable shaft is being rotated, or when the rotatable shaft is not rotated. In other embodiments, the head may comprise multiple points or edges that may be used to cut, chop, grind, burr, pulverize, debride, debulk, emulsify, disrupt or otherwise remove tissue or body structures. In still other embodiments, the head may comprise surfaces with a grit that may be used as a burr mechanism. The grit number may range from about 60 to about 1200 or more, sometimes about 100 to about 600, and other times about 200 to about 500.

The head may optionally comprise a port or aperture which may be used to perform suction or aspiration at the target site and/or to perfuse saline or other biocompatible fluids or materials to the target site. Use of saline or other cooling materials or liquids, for example, may be used to limit any thermal effect that may occur from frictional or other forces applied to the target site during removal procedures. The saline or other materials may or may not be chilled. In other embodiments, one or more therapeutic agents may be provided in the saline or fluid for any of a variety of therapeutic effects. These effects may include anti-inflammatory effects, anti-infective effects, anti-neoplastic effects, anti-proliferative effects, hemostatic effects, etc.

FIG. 9 depicts impeller 804 in additional detail. As shown there, impeller 804 resembles a parabolic drill bit, with a proximal end 902, a distal end 904, and a relatively large relief cut into the back section of its flutes to allow for clearance and better flow of material as the material is drilled out. The impeller 804 also may have forward cutting abilities with a pointed distal end 904 and/or open flutes at the distal end or tip 904, which may aid in centering the impeller. Impeller 804 may, for example, have a pitch (or distance between two adjacent revolutions of the impeller) of about 0.2 inch to about 0.4 inch (e.g. 0.3 inch). In certain embodiments, the length of the helical path of the impeller may be about 0.5 inch to about 2.0 inches (e.g. about 0.7 inch to about 1.5 inches or about 1 inch to about 1.3 inches, such as 1.12 inches). Impellers may be made of any appropriate material or materials, including but not limited to metals and/or metal alloys such as stainless steel (e.g. 17-4 PH H900 stainless steel).

It should be understood that features of the above-described impellers and/or other components described herein may be applied to other impellers and/or components of tissue removal devices, as appropriate.

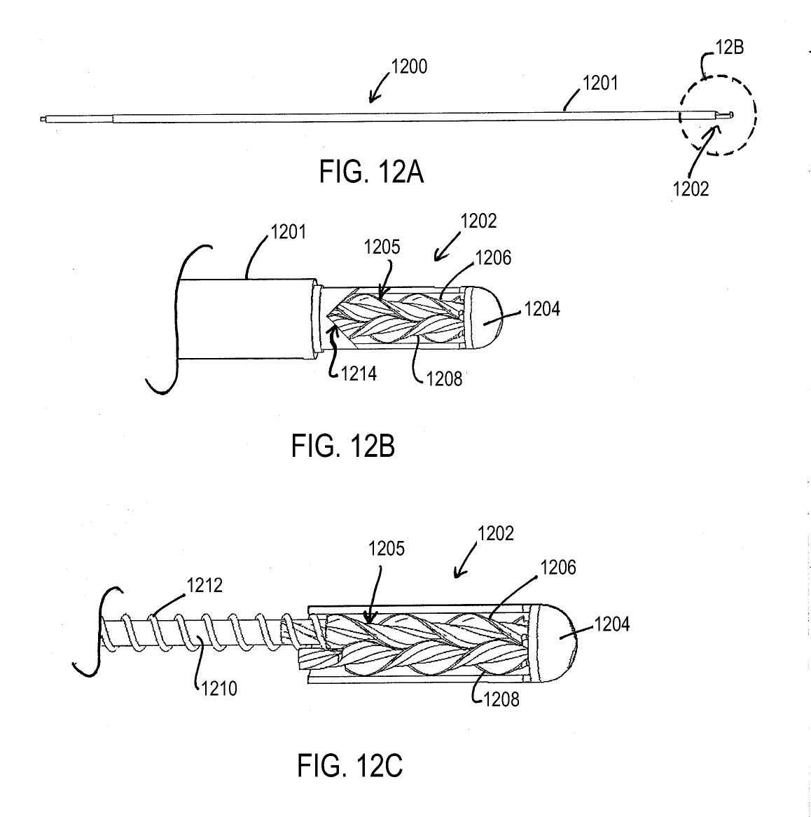

In some cases, a tissue removal device may comprise multiple impellers. As an example, FIG. 12A shows a shaft 1200 of a tissue removal device comprising a tissue removal assembly 1202. As shown in FIG. 12B, the tissue removal assembly 1202 comprises a hooded tip or housing 1204 having an aperture 1205 therein and housing two impellers 1206 and 1208. In some cases, the hooded tip 1204 may be formed of one or more metals and/or metal alloys (e.g. stainless steel), and the hooded tip 1204 may be soldered to another component of the tissue removal device, such as a tubular member 1201. Other couplings between the hooded tip and one or more other device components may alternatively or additionally be used.

As shown in FIGS. 12B and 12C, the impellers 1206 and 1208 are similar in configuration to parabolic drill bits, and may be configured to counter-rotate relative to each other. The impeller 1206, which has a right-hand spiral, and the impeller 1208, which has a left-hand spiral, may be configured to nest together and overlap in a way that maximizes tissue contact with the impellers' shearing edges. During use, the impeller 1208 may rotate freely, but may be constrained axially (e.g. by the hooded tip 1204 and/or one or more other components of the tissue removal device). Rotation of the impeller 1208 may, for example, be driven purely by rotation of the impeller 1206.

While impellers having a certain configuration have been described, it should be understood that other configurations of impellers or tissue removing cutters having one or more shearing edges may alternatively or additionally be used. Moreover, more than two (e.g. three, four, five) impellers may be used in some cases. The impellers may all have the same size and/or shape, or at least some of the impellers may have different sizes and/or shapes. Furthermore, the impellers need not necessarily be positioned adjacent one another, in a side-by-side fashion. For example, in some cases one impeller may be positioned on top of another impeller. Additionally, in certain embodiments, impellers may not be nested together and/or may not overlap with each other. Furthermore, in some embodiments, a tissue removal device may comprise multiple impellers, at least two of which are configured to rotate independently of the other impellers.

Referring again to FIG. 12C, one impeller 1206 is coupled to a rotating shaft 1210 and helical auger 1212 of the tissue removal assembly 1202. Thus, the impeller 1206 may rotate along with the rotating shaft 1210 (e.g. controlled by a motor in the handle of the tissue removal device, such as a motor described herein). While the other impeller 1208 is not coupled to the rotating shaft 1210 or helical auger 1212, some embodiments of tissue removal devices may comprise multiple impellers coupled to a rotating shaft and/or auger.

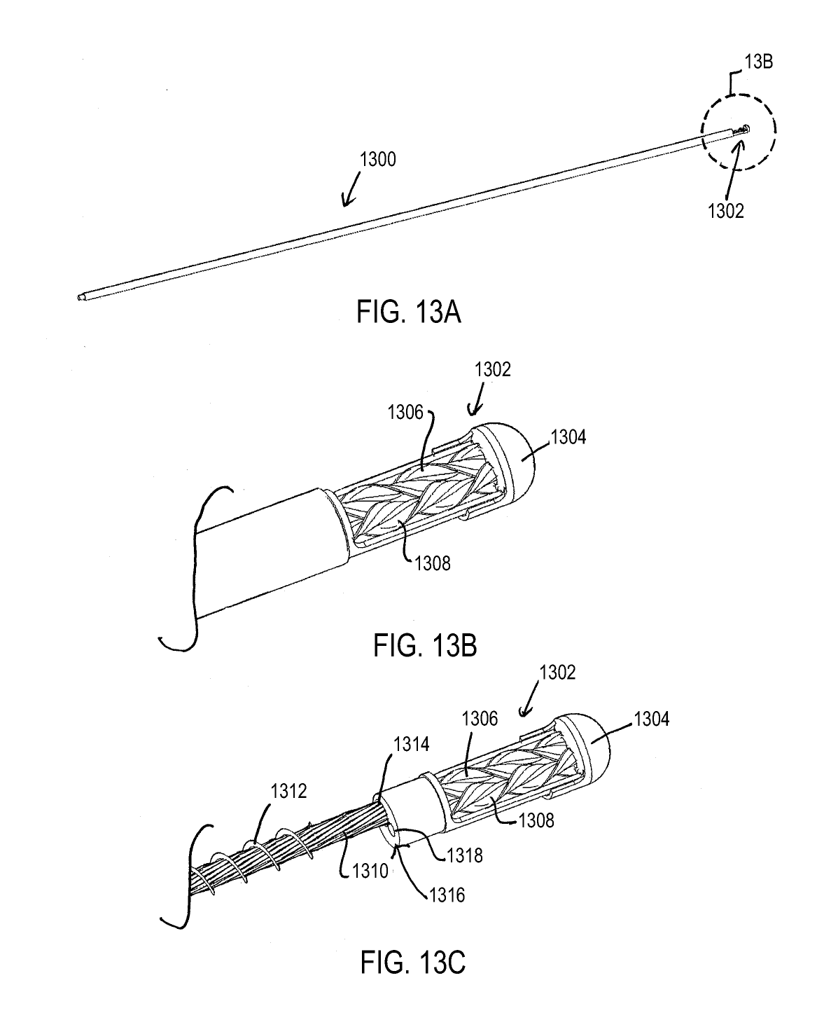

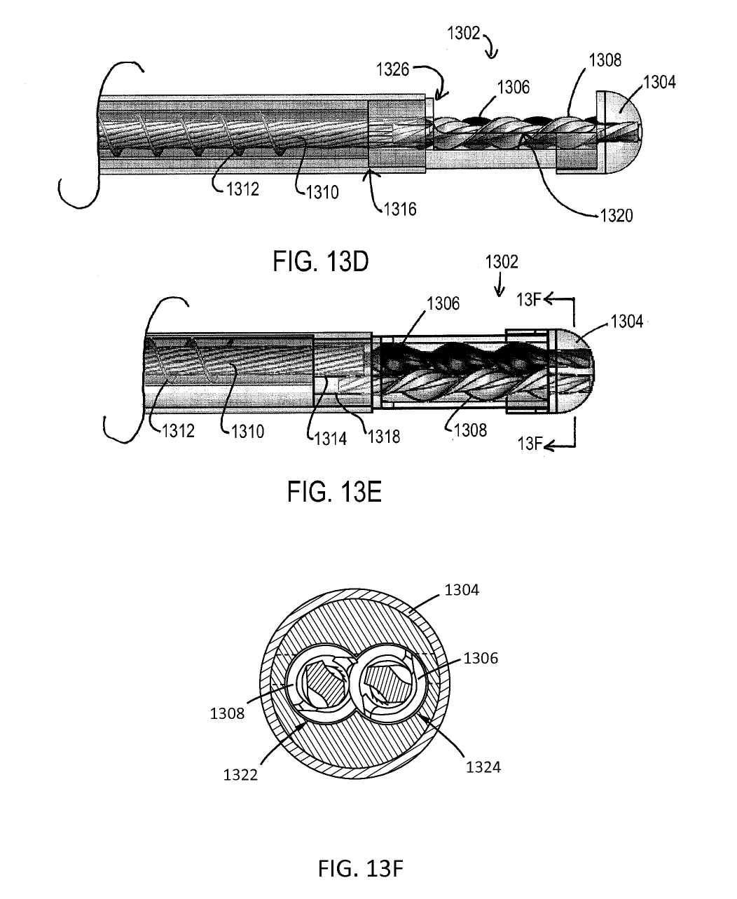

Referring to FIG. 12B, hooded tip 1204 includes a V-shaped proximal shearing edge 1214, which is generally angled counter to the helical shape of the impellers 1206 and 1208. However, some embodiments of tissue removal devices may comprise tissue removal assemblies including multiple shearing edges, while other embodiments of tissue removal devices may comprise tissue removal assemblies having no shearing edges. Shearing edges may be arranged at any suitable angle. FIG. 13A shows a shaft 1300 of a tissue removal device comprising a tissue removal assembly 1302 including a hooded tip or housing 1304. Two impellers 1306 and 1308 are disposed within the hooded tip 1304. As shown in FIGS. 13B-13E, hooded tip 1304 does not include any shearing edges. Additionally, as shown in FIGS. 13C-13E, a helical auger 1312 is coupled to a rotating shaft 1310 that passes through an aperture 1314 in a proximal end 1316 of hooded tip 1304. Proximal end 1316 further includes a second aperture 1318 that may be used, for example, to stabilize the impeller 1308. FIG. 13F provides a cross-sectional view of the hooded tip 1304, taken along line 13F-13F in FIG. 13E.

The hooded tip or housing 1304 may have a relatively large opening 1320 (FIG. 13D) to accommodate both impellers 1306 and 1308. For example, the opening may have a wrapped dimension (i.e. a measurement taken along the circumference of the hooded tip 1304) of at least about 0.050 inch and/or a length of at least about 0.150 inch. Additionally, the hooded tip 1304 may include two separate grooves 1322 and 1324 (FIG. 13F) to, for example, provide relatively tight clearance between each impeller 1306 and 1308 and the hooded tip or housing 1304. This relatively tight clearance may, for example, result in improved shearing efficiency. It should be understood that such grooves are optional, and some embodiments of devices may not include any grooves, while other embodiments of devices may include more than two grooves. As shown in FIG. 13E, the impellers 1306 and 1308 are also positioned in distal holes or indentations 1352 and 1354 provided in hooded tip or housing 1304. The holes or indentations 1352 and 1354 may be formed, for example, by drilling. Of course, other embodiments of hooded tips may alternatively or additionally include one or more other impeller retention features, such as bumps, hooks or the like.

In some cases, one or more additional shearing edges having an angled geometry (not shown) may be added to the hooded tip or housing 1304 (e.g. to its proximal side 1326 (FIG. 13D)). Such a shearing edge may, for example, have a configuration similar to the V-shaped proximal shearing edge 1214 of the hooded tip 1204 shown in FIG. 12B. While not depicted, in some cases a hooded tip may comprise one or more distal shearing edges.

Referring back to FIGS. 4B and 4E, tissue removal device 400 is illustrated with a portion of the housing 406 removed to show various internal components. In this embodiment, the tissue removal device 400 further comprises a battery 450 to provide power to a motor 452 which drives the tissue removal assembly 408. In other embodiments, a connector to an external power source may be provided in addition to, or in lieu of, the battery 450. The type of battery and power provided may differ depending upon the particular power needs of the motor and/or other components of the tissue removal device 400.

In some embodiments, the motor 452 of the tissue removal device 400 is a DC motor, but in other embodiments, the motor 452 may have any of a variety of configurations, including but not limited to an AC or a universal motor. The motor 452 may be a torque, brushed, brushless or coreless type of motor. In certain embodiments, the motor 452 may be configured to provide a rotational speed of about 500 rpm to about 200,000 rpm or more, sometimes about 1,000 rpm to about 40,000 rpm, and other times about 5,000 rpm to about 20,000 rpm. The motor 452 may act on the tissue removal assembly 408 via the shaft 404, or by a drive member located within shaft 404. In some further embodiments, a fluid seal may be used to protect the motor 452 and/or other components of the housing 406 from any fluids or other materials that may be transported through shaft 404. In certain embodiments, housing 406 may be configured to be coupled to a trocar, an introducer, a cannula or another tubular member into which the tissue removal assembly 408 and the shaft 404 are inserted during use. In some embodiments, the tissue removal device may be used with an introducer or cannula having an outer diameter of about 0.1 cm to about 1.5 cm or more, sometimes about 0.1 cm to about 1 cm, and other times about 2 mm to about 6 mm.

In certain embodiments, a housing of a tissue removal device, such as housing 406, may be configured with a size and/or shape that permits handheld use of the tissue removal device. In other embodiments, the tissue removal device may comprise a grip or structure (e.g. located about a tubular member of the device) to facilitate handling by the user, while a proximal end (e.g. of the tubular member) may be attached to a benchtop or cart-based machine, for example, or a mounted or fixed machine. In these embodiments, the grip may or may not contain any other components of the tissue removal device, such as a motor, while the machinery at the proximal end (e.g. of the tubular member) may contain one or more other components, such as a suction system or various radiofrequency ablation components, for example. In some embodiments, the housing may have a length of about 1 cm to about 12 cm or more, sometimes about 2 cm to about 8 cm, and other times about 3 cm to about 5 cm. The average diameter of the housing (or other transverse dimension to the longitudinal axis of the housing) may be about 1 cm to about 6 cm or more, sometimes about 2 cm to about 3 cm, and other times about 1.5 cm to about 2.5 cm. The housing may further comprise one or more ridges, recesses or sections of textured or frictional surfaces, including but not limited to styrenic block copolymers or other polymer surfaces.

Some embodiments of tissue removal devices described herein may also be capable of aspirating tissue. For example, a tissue removal device may comprise a conduit which may be used to connect the tissue removal device to an aspiration or suction source. An aspiration or suction source may be used, for example, to transport fluid or material through a lumen or conduit of a tubular member of the tissue removal device. In certain embodiments, one or more separate ports may be provided for infusing or injecting substances into a target site using the tissue removal device. In other embodiments, the above-described conduit may be used for both withdrawal and infusion of materials and/or fluids, or for infusion only. Depending upon the configuration of the tissue removal device, withdrawal and/or infusion may occur at the distal end of the device, and/or through one or more openings of the tissue removal assembly of the device. In some embodiments, a port may be used to insert a coagulation catheter, an ablation catheter, or another energy delivery device to the target site.

The various tissue removal devices disclosed herein may be used to perform a discectomy or nucleotomy, but may also be used to perform any of a variety of tissue removal procedures in the spine and outside of the spine. Examples of procedures that may be used to access the spine are disclosed in U.S. Pat. No. 7,108,705, U.S. Pat. No. 4,573,448, U.S. Pat. No. 6,217,509, and U.S. Pat. No. 7,273,468, which are hereby incorporated by reference in their entirety.

The tissue removal devices may be used in minimally invasive procedures as well as open surgical procedures or limited access procedures. These procedures may include but are not limited to interlaminar, translaminar and intralaminar access procedures. In one particular embodiment, a patient may be placed into a prone position with a pillow or other structure below the abdomen to limit lumbar lordosis. The patient may be prepped and draped in the usual sterile fashion and anesthesia may be achieved using general, regional or local anesthesia. Under fluoroscopic guidance, a sharp tipped guidewire, or a needle with a guidewire, may be inserted into the paravertebral space or epidural space from a posterior or postero-lateral location of the patient's back at a location in the range of about 2 inches to about 6 inches lateral to the midline. In some instances, guidewire insertion may be facilitated by inserting a needle into the tissue first. In alternate embodiments, an anterior procedure through the abdominal cavity or anterior neck region may be performed. Once access to the target location is confirmed, a dilator may be used with the guidewire to enlarge the insertion pathway. Then, an introducer or cannula may be inserted over the guidewire, followed by subsequent guidewire removal and insertion of an endoscope into the introducer or cannula. Alternatively, an endoscope may be inserted over the guidewire. The endoscope may be manipulated or steered to directly visualize and identify the relevant structures such as the disc, the nerve or other adjacent structures and site(s) of tissue removal. In some embodiments where the patient is under local or regional anesthesia, a suspected nerve impingement may be confirmed by contacting or manipulating the suspected nerve with the endoscope, or other device inserted through the endoscope, and assessing the patient's response or symptoms. One embodiment of an endoscope that may be used is described in U.S. patent application Ser. No. 12/199,706, which is hereby incorporated by reference in its entirety.

Once the target region has been evaluated, a tissue removal device may be inserted through the spinal access device or endoscope and to pierce through the annular wall of a herniated disc. Once inserted, the tissue removal device may be manipulated and actuated to remove the target tissue. In some embodiments, the tissue removal device may be actuated for a duration in the range of about 5 seconds to about 90 seconds or more, sometimes about 15 seconds to about 60 seconds, and other times about 30 seconds to about 60 seconds.

In certain embodiments, any collected material may be suctioned through the device and then the effect of the tissue removal may be re-evaluated by the endoscope or other visualization mechanisms. In some embodiments, a liquid or lubricant may be injected or infused into the treatment site. In some examples, the liquid or lubricant may be useful to facilitate removal of the collected material, including but not limited to vertebral discs that may be desiccated. In other examples, the liquid or lubricant may be injected or infused before or during the actuation of the tissue removal device. In some examples, the liquid or lubricant may comprise a contrast agent that may facilitate viewing of the tissue site on fluoroscopy, X-ray, CT, MRI, ultrasound or other imaging modalities. The contrast agent may be used at any time or at multiple times during the procedure, including but not limited to confirmation of guidewire or tissue removal device placement, and also to verify the volume and/or location of tissue removal.

In some specific embodiments, actuation of the tissue removal device may be stopped to verify that the annulus of the vertebral disc or the cortical bone of the vertebral body has not been compromised. Also, in some examples, contrast agent may be injected and imaged after device actuation to assess proper operation of the device, including but not limited to tissue pulverization and aspiration mechanisms.

During actuation, the tissue removal device may be held in place or may be moved around the treatment site. Suction or aspiration may be applied during these motions to assess the amount of tissue being removed.

The actuation of the tissue removal device may be repeated as desired to remove disc material. In some embodiments, the tissue removal device may be withdrawn from the disc and reinserted directly into or against the extruded disc material and actuated. Once the tissue removal is completed, the tissue removal device may be withdrawn. The puncture site in the annular wall may have a cross-sectional area of less than about 0.003 inch² or less, sometimes about 0.0016 inch² or less, and other times about 0.001 inch² or less, and thus may self-seal without requiring treatment of the puncture location with an adhesive, a suture or coagulation probe. The body location may be rechecked with the endoscope or spinal access device to verify that no bleeding or compromise of the integrity of the disc or spinal nerves has occurred, and then the endoscope or spinal access device may be removed from the body and the skin access site may be bandaged.

While various tissue removal devices may be used to remove larger volumes of tissue, in other embodiments, a tissue removal device may be used to perform focal debulking of tissue. For example, by utilizing the small profile and/or the steerable features of certain embodiments of the tissue removal device, the tissue removal device may be more accurately positioned or navigated to a specific target site in a body structure. In some instances, the removal of lower volumes of tissue at a specific target location may be used to achieve a desired result, in comparison to the removal of a larger volume of tissue from a general target location. By removing less disc tissue to reduce a herniation, for example, a larger amount of non-pathologic disc tissue and structural integrity of the disc may be preserved. In some instances, relatively greater preservation of the disc tissue may slow the rate of further disc degeneration and reherniation compared to lesser degrees of tissue preservation.

In one example, a herniated disc may be accessed and visualized endoscopically. A steerable tissue removal device may be inserted into the disc and steered toward the region of herniation, rather than to the center of the disc, for example.

The procedures described herein may target vertebral tissue in different locations, and as such, access sites and pathways may vary accordingly. The tissue removal devices described above may be used with one or more access devices which may help direct the tissue removal device to the target tissue site. An access device, such as a cannula, may be positioned with different angles of entry depending on the location of the targeted vertebral tissue. The range of suitable entry angles may be at least partially constrained by the location of spinal structures with respect to the skin surface. For example, a straight cannula may be positioned within the range of suitable entry angles to create a linear access pathway that extends from an access site on the skin surface to a targeted region of spinal tissue that is co-linear with access site. A curved cannula may be used to create a curved pathway to access tissue that may not be co-linear with an access site within a suitable entry angle range. While a curved pathway may provide increased accessibility to vertebral tissue, a practitioner may need to undergo additional training and practice to avoid disrupting sensitive anatomical structures along a curved pathway. Some embodiments of access devices may comprise a bendable flexible curvable cannula, which may have a straight configuration and a curved configuration. The cannula may be used in the straight configuration to create a substantially linear access pathway from the access site on the skin surface to the vicinity of the target vertebral tissue. Once the initial access pathway is created, the cannula may be used in the curved configuration to contact the target tissue.

In some embodiments, the curvature of a cannula may be determined in part by the curvature of a stylet inserted therethrough. For example, inserting a stylet with one or more curves into a bendable flexible cannula may cause the cannula to have corresponding curves. In some embodiments, a bendable cannula may have one or more pre-formed curves that may be straightened by inserting a straight stylet therethrough. Alternatively, a bendable cannula that is substantially straight may be curved by inserting a curved stylet therethrough. The insertion of various stylets through a bendable cannula may allow a practitioner to access spinal tissue at different locations via one access site on the skin. This may reduce the need for withdrawing the cannula from the body and re-entering the body via an additional access site to access a different tissue region. For example, the cannula and the stylet may each have one or more corresponding curves such that when the stylet is inserted through the cannula, the corresponding curves may be aligned. This may act to stiffen or reinforce the curvature of the cannula so that it may be more easily moved from a first tissue location to a second tissue location. For example, a procedure performed on one tissue location in the disc annulus may be repeated at another tissue location without removing the curved cannula from the disc annulus. While at the first tissue location, a curved or straight stylet may be reintroduced into the cannula, which may facilitate adjustment and positioning of the cannula to a second tissue location. Insertion of a straight stylet may straighten the curved portion of the cannula and allow the cannula-stylet assembly to be advanced to a target site that is relatively further away from the site that has been treated. In other embodiments where relatively insignificant cannula repositioning is involved, a curved stylet may be used to acquire access to a second target site within the disc. A straightened and/or stiffened cannula-stylet assembly may offer enhanced responsiveness and maneuverability and therefore facilitate the maneuvering of the cannula within the discal area, and may facilitate safe removal of the devices from a patient.

The length of a stylet may be greater than, or substantially equal to the length of a corresponding cannula. For example, the distal portion of a stylet inserted into a cannula may extend or protrude from the distal portion of the cannula, and/or may be flush with the distal portion of the cannula, and/or may even be withdrawn into the cannula, as desirable. Similarly, the tissue removal assembly of a tissue removal device may be extended from and/or withdrawn into the distal portion of the cannula. The relative longitudinal position between a cannula and stylet, and/or cannula and a travel limiter of a tissue removal device may be adjusted and/or locked. In some embodiments, the orientation of one or more curves in a cannula and a stylet with respect to each other may be adjusted by rotating the stylet, and may optionally be locked once the desired orientation is obtained. The cannula and stylet may each comprise complementary proximal connectors, which may be used to couple them together, such that they may be advanced and navigated together. Optionally, the proximal connectors may rotatably and/or longitudinally lock the cannula and stylet with respect to each other.

Some embodiments of a cannula and/or stylet may have an orientation indicator, which may help a practitioner to identify the orientation of the one or more curves of the devices, or the orientation of one or more sharpened edges of a stylet, after they have been inserted into the body of a patient. For example, the orientation of a distal curve of a cannula with respect to the longitudinal axis of the cannula shaft may be evident by observing the configuration of the orientation indicator. Orientation indicators may also help a practitioner align the curvature of a stylet to correspond with the curvature of the cannula that it is inserted through. In this way, the practitioner may proximally adjust the bend orientation of the stylet, thereby allowing the stylet to pass through the cannula bend with ease. The shape of the orientation indicator may convey the orientation of the one or more curves of the cannula and/or style to the practitioner. For example, the orientation indicator may have a shape with one or more tapered regions, where the plane of a taper is indicative of the plane of a distal curve. In some embodiments, orientation indicators may have multiple apices that are aligned with multiple curves in multiple planes, which may help the practitioner position and orient the distal portion of the tissue removal device as desired. The orientation indicator may be attached to the cannula and/or stylet by soldering, welding, adhesive bonding (e.g. 3311 UV adhesive that may be UV cured), snap fit, or other appropriate methods. In some embodiments, the orientation indicator may be attached or integrally formed with a proximal connector of the cannula and/or stylet. This may provide a mechanism for the cannula and stylet to be coupled together in a particular orientation.

Cannulas and stylets may each have proximal connectors that couple them to each other. The proximal connector of a cannula may also be used to couple it with a tissue removal device, for example, a collector port and/or travel limiter. Connectors may be any standardized connector (e.g. any luer-type connectors, screw-type connectors, taper ground joints, etc.), or may be a proprietary connector. In some embodiments, a cannula may have a male-type connector that is configured to connect with a stylet or tissue removal device with a female-type connector. Engagement of the proximal connectors of cannula, stylets, and/or tissue removal devices may prevent relative movement between the devices. In some embodiments, when a stylet is connected to a cannula, the stylet may not be able to move longitudinally within the cannula, but may be axially rotated within the cannula. This may allow a practitioner to adjust the alignment between the cannula and stylet during the insertion of the cannula and stylet into the body. Alternatively or additionally, engagement of the proximal connectors between a cannula and stylet, or a cannula and a travel limiter of a tissue removal device may prevent relative longitudinal and axial motion between the devices. Locking the orientation and position between the cannula and stylet (and/or cannula and travel limiter) may help prevent inadvertent device misalignment or movement during a procedure. Travel limiters are disclosed, for example, in U.S. Patent Application Ser. No. 61/425,226, which is incorporated herein by reference in its entirety.

In some examples, the distal region of the cannula and/or stylet may comprise a radio-opaque structure (e.g. rings or bands) to facilitate confirmation of its position using radiographic imaging. In other examples a separate radiographic marker instrument may be used to confirm and evaluate the cannula placement.

In some embodiments, a bendable flexible curved cannula may be used in association with either a straight stylet or a curved stylet to obtain curved access to a spinal area. A curved access pathway not only offers a larger tissue removal zone at one target site, but it may also provide flexible access to multiple target sites in one or more herniated discs. A curved or non-linear access pathway that may be provided by a bendable flexible curved cannula may be shorter than a straight access pathway, and may be less disruptive to surround tissue structures. It may also provide better orientation towards the middle of a disc, as compared with a straight access pathway.

The bending range of the curved cannula may be in the range of from about 10 degrees to about 80 degrees, sometimes from about 20 degrees to about 70 degrees, and other times from about 30 degrees to about 60 degrees, and still other times from about 40 degrees to about 50 degrees. The curved distal portion may comprise a radius of curvature of about 0.5 centimeter to about 30 centimeters; sometimes about 1 centimeter to about 20 centimeters, sometimes about 5 centimeters to about 15 centimeters and other times about 8 centimeters to about 10 centimeters. When the curved distal portion is straightened, the curved cannula may comprise a length of about 4 inches to about 12 inches or more, sometimes about 5 inches to about 10 inches, and other times about 6 inches to about 9 inches.

Prior to inserting the tissue removal device into the cannula, approximately 0.5 cc of saline may be injected into the disc through the cannula. Under image guidance, the tissue removal device may be inserted through the cannula until the target site has been reached. Using image guidance, the practitioner may advance the tip of the tissue removal device to the full plunge depth, and confirm that the tip is in a safe location. The tissue removal device may then be actuated. The placement of the device in the course of tissue removal may be intermittently confirmed by fluoroscopy or another appropriate imaging modality. The tissue removal device may be used until sufficient tissue material has been removed, and/or the collector is full. In some embodiments, a negative pressure source may be coupled to the collector which may help expedite tissue removal. The markings on the collector indicate the quantity of tissue removed. The tissue removal device may be turned on and used continuously for about 0.5 second to about 6.0 minutes, e.g., 2.0 minutes.

Once a sufficient quantity of tissue material has been removed, the tissue removal device may be turned off. The above steps may be repeated until the desired quantity of tissue has been removed. If additional treatment is required within the disc, the straight or curved stylet may be reinserted into the cannula, and the cannula may be repositioned. In some procedures, it may be desirable to limit the total run-time of the tissue removal device to about 6.0 minutes or less. The straight stylet may be inserted into the cannula and fixedly attached at the proximal hub. Then, the cannula-straight stylet assembly may be withdrawn from the access site. In some embodiments, the battery of the tissue removal device may be removed and disposed according to local regulations.

The cannula, stylet, and tissue removal devices described above may be used to perform a discectomy. The devices may be used in a minimally invasive procedure, or an open surgery procedure. The cannula-stylet assembly may be used to form a passageway or a working channel through the tissue about a target site in the spinal region. For example, to perform a discectomy procedure, the patient may be prepped and draped in the usual sterile fashion and in a lateral decubitis or prone position. General, regional or local anesthesia may be achieved. A straight stylet with a sharp distal tip may be inserted into the lumen of a straight cannula. The assembly may then be percutaneously inserted through a posterior or posterolateral entry point on the back of the patient. The cannula-stylet assembly may be further inserted into the epidural space or into the paravertebral space, depending on the assembly's point of entry. Alternatively, the assembly may be used to penetrate the disc annulus directly from a point of entry further away from the midline of the patient's back. In some embodiments, the assembly may be introduced on the ipsilateral side from which the nerve impingement has been identified and at an angle of about 25 degrees to about 45 degrees to the patient's back. In other procedures, a contralateral approach and/or a different angle may be used. In alternative embodiments, an anterior procedure through the abdominal cavity of the anterior neck region may be performed.

The cannula-stylet assembly may be advanced together to a target tissue site, as described above. During the insertion of the assembly, the stylet may be independently rotatable such that the operator may adjust the orientation of the optional beveled edge of the stylet in order to form a passageway through the surrounding tissue, bones or other anatomic structures. The insertion of the cannula-stylet assembly may be performed under the guidance of external imaging and/or visualization techniques.

Fluoroscopy and/or CT scan may be used before, during and/or after the procedure to assess the patient's anatomy, the position of the instruments, the structural changes after tissue removal, and/or to verify the integrity of the disc. In some embodiments, a small amount of radiopaque contrast agent may be injected into the disc space to enhance visualization. Such injection may be performed by the tissue removal device through an infusion or irrigation channel, or through the aspiration port. In other embodiments, the cannula may comprise an infusion or irrigation lumen to introduce the contrast agents. In some embodiments, the tissue removing procedure may be assessed by the quantity and/or color of the tissue removed through an optically transparent chamber, or collection chamber. Upon completion of the procedure, the tissue removal device may be proximally withdrawn, followed by withdrawal of the cannula.

Devices described herein may be used with one or more visualization systems, such as one or more endoscopic visualization systems, as appropriate.

It is to be understood that this invention is not limited to particular exemplary embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a blade” includes a plurality of such blades and reference to “the energy source” includes reference to one or more sources of energy and equivalents thereof known to those skilled in the art, and so forth.

The publications discussed herein are provided solely for their disclosure. Nothing herein is to be construed as an admission that the present invention is not entitled to antedate such publication by virtue of prior invention. Further, the dates of publication provided, if any, may be different from the actual publication dates which may need to be independently confirmed.

What is claimed is:

1. A tissue removal device comprising:
a handheld housing;
a motor; and
a tissue removal mechanism coupled to the handheld housing, the tissue removal mechanism comprising:
a tubular member;
a rotatable elongated member disposed within a lumen of the tubular member;
a first impeller distal to the rotatable elongated member; and
a second impeller adjacent the first impeller.

2. The tissue removal device of claim 1, wherein the second impeller is configured to counter-rotate with respect to the first impeller.

3. The tissue removal device of claim 1, wherein rotation of the rotatable elongated member effects rotation of the first impeller.

4. The tissue removal device of claim 3, wherein rotation of the first impeller effects rotation of the second impeller.

5. The tissue removal device of claim 3, further comprising a helical member disposed around at least a portion of the rotatable elongated member.

6. The tissue removal device of claim 5, wherein rotation of the rotatable elongated member also effects rotation of the helical member.

7. The tissue removal device of claim 1, further comprising an impeller housing, wherein the first and second impellers are disposed within the impeller housing.

8. The tissue removal device of claim 7, wherein the impeller housing comprises a side wall portion including a first aperture therethrough.

9. The tissue removal device of claim 8, wherein the side wall portion further includes a second aperture therethrough.

10. The tissue removal device of claim 9, wherein the first and second apertures are configured to expose the first and second impellers to tissue during use.

11. The tissue removal device of claim 9, wherein at least one of the first and second apertures defines a cutting edge.

12. The tissue removal device of claim 11, wherein the cutting edge has a serrated configuration.

13. The tissue removal device of claim 7, further comprising a sheath disposed within the impeller housing, wherein the first and second impellers are disposed within the sheath.

14. The tissue removal device of claim 1, further comprising a tissue collection chamber coupled to a distal portion of the handheld housing.

15. A tissue removal device comprising:
a handheld housing;
a motor; and
a tissue removal mechanism coupled to the handheld housing, the tissue removal mechanism comprising:
a tubular member;
a rotatable elongated member disposed within a lumen of the tubular member;
an impeller housing coupled to a distal end of the tubular member; and
an impeller disposed within the impeller housing and coupled to the rotatable elongated member,
wherein the impeller housing comprises a side wall portion having first and second apertures therethrough, and wherein the first and second apertures are configured to expose the impeller to tissue during use.

16. The tissue removal device of claim 15, wherein at least one of the first and second apertures defines a cutting edge.

17. The tissue removal device of claim 16, wherein the cutting edge has a serrated configuration.

18. The tissue removal device of claim 15, wherein the first aperture is opposite the second aperture along a circumference of the impeller housing.
